(19) United States
(12) Patent Application Publication
(10) Pub. No.: US 2025/0017969 A1
(43) Pub. Date: Jan. 16, 2025

(54) PREPARATION OF ENGINEERED MYELOID CELLS

(71) Applicant: VITA THERAPEUTICS, INC., Baltimore, MD (US)

(72) Inventors: Aihong ZHANG, Baltimore, MD (US); Amir SABERI, Baltimore, MD (US); Douglas FALK, Baltimore, MD (US); Peter ANDERSEN, Baltimore, MD (US); Daniel J. GRUN, Baltimore, MD (US)

(21) Appl. No.: 18/713,136

(22) PCT Filed: Nov. 28, 2022
(86) PCT No.: PCT/US2022/080502

§ 371 Date: May 23, 2024

Related U.S. Application Data
(60) Provisional application No. 63/283,384, filed on Nov. 26, 2021.

(51) Int. Cl. A61K 35/17; A61K 39/00; C07K 14/705; C12N 5/0786

(52) U.S. Cl. CPC A61K 35/17; A61K 39/4614; A61K 39/464429; C07K 14/70521; C07K 14/70596; C12N 5/0645; C07K 2319/03; C07K 2319/33; C12N 2500/02; C12N 2510/00

(57) ABSTRACT

The present disclosure provides methods for preparing immune cells that express a chimeric receptor. The immune cells, preferably p50 deficient immature myeloid cells, exhibited improved therapeutic efficacy as compared to the conventional immune cell therapies and are more broadly applicable to different types of cancers. The preparation methods preferably include inactivating p50 in a progenitor cell, expanding the cell under hypoxic conditions, transducing a polynucleotide that encodes the chimeric receptor, and differentiating the engineered progenitor cell to an immature myeloid cell.

### CROSS REFERENCE TO RELATED APPLICATIONS

This application claims the benefit under 35 U.S.C. § 119 (e) of the U.S. Provisional Application Ser. No. 63/283,384, filed Nov. 26, 2021, the content of which is hereby incorporated by reference in its entirety.

### REFERENCE TO AN ELECTRONIC SEQUENCE LISTING

The contents of the electronic sequence listing (66CM-345111-WO; Size: 53,709 bytes; and Date of Creation: Nov. 28, 2022) is herein incorporated by reference in its entirety.

### BACKGROUND

Chimeric antigen receptor T cells (also known as CAR T cells) are T cells that have been genetically engineered to produce an artificial T cell receptor for use in immunotherapy. CAR-T therapy has the potential to improve the management of lymphomas and possibly solid cancers. A number of CAR-T products have been approved by the FDA for the treatment of cancer, including those targeting CD19 or the B-cell maturation antigen (BCMA).

The efficacy of the current CAR-T therapies for treating solid cancer, however, is limited by several factors, including the inherent heterogeneity of the cancer cells, strong immunosuppressive tumor microenvironment (TME) contributed by myeloid-derived suppressor cells (MDSC) and/or tumor promoting M2 macrophages, and the lack of tumor penetration by the adoptive transferred immune cells.

Further, manufacturing, in particular expansion, of CAR-T cells is challenging. There are, therefore, unmet needs in the art for improved cell therapy compositions and methods for their preparation.

### SUMMARY

The present disclosure, in various embodiments, provides methods for preparing recombinant immune cells suitable for cellular therapies. In some embodiments, the immune cells are p50 deficient immature myeloid cells. The preparation methods, in some embodiments, include inactivating p50 in a progenitor cell, expanding the cell under hypoxic conditions, transducing a polynucleotide that encodes the chimeric receptor, and differentiating the engineered progenitor cell to an immature myeloid cell.

In accordance with one embodiment of the present disclosure, provided is a method for preparing an immune cell that expresses a chimeric receptor, which comprises introducing to a progenitor cell or an immune cell a polynucleotide that encodes a chimeric receptor. In some embodiments, the chimeric receptor comprises, from the N-terminus to the C-terminus, an extracellular domain of a receptor to a tumor-associated ligand, a transmembrane domain, a costimulatory domain, and a CD3ζ intracellular domain.

In some embodiments, prior to or following the transduction, the progenitor cell or immune cell is cultured or expanded in a medium under a hypoxic condition. In some embodiments, the hypoxic condition is induced by a cobalt salt in the medium, such as 20 μM to 200 μM CoCl₂, or preferably 50 μM to 150 μM CoCl₂, in the medium. Alternatively, in some embodiments, the hypoxic condition is induced by placing the medium in a chamber having no more than 10% oxygen in the air, preferably no more than 5%, or 2% or 1% oxygen in the air.

The progenitor cell may be a CD34⁺ cell. The immune cell, in some embodiments, is an immature myeloid cell. In some embodiments, the progenitor cell or immune cell is p50 deficient.

Example progenitor cells included, without limitation, a CD34⁺ hematopoietic stem cell from bone marrow, a mobilized CD34⁺ hematopoietic stem cell from peripheral blood, and an induced pluripotent stem cell (iPSC). In some embodiments, the progenitor cells are CD34⁺ hematopoietic stem cell from bone marrow.

The progenitor can be differentiated into the immune cell, prior to or following the transduction. In some embodiments, the differentiation is carried out in a differentiation medium comprising stem cell factor (SCF), thrombopoietin (TPO) and/or FMS-like tyrosine kinase 3 ligand (FLT3L). Subsequently, in some embodiments, the differentiation is in a medium that comprises macrophage colony-stimulating factor (M-CSF).

In some embodiments, the extracellular domain in the chimeric receptor is a ligand-binding domain of a natural receptor. In some embodiments, the natural receptor is selected from the group consisting of PD1, SIRPα, Siglec-10, CTLA-4, CXCR-4, CCR-2, CXCR2, CCR7, CD80, TIM-3, LAG3 and TREM2. In some embodiments, the receptor is PD1. In some embodiments, the costimulatory domain is a signaling domain of a protein selected from the group consisting of CD28, CD27, CD40, CD80, CD86, OX40, and 4-1BB.

In some embodiments, the immune cell further comprises exogenous polynucleotide encoding a proinflammatory cytokine. In some embodiments, the proinflammatory cytokine is selected from the group consisting of IL-12, IFN-γ, TNF-α, and IL-1β. In some embodiments, the immune cell further comprises a kill switch. In some embodiments, the kill switch is selected from the group consisting of HSV-TK, truncated EGFR (tEGFR), and CD20.

In a specific example embodiment, the instant disclosure provides a method for preparing an immune cell, comprising: decreasing the biological activity of the p50 gene in a progenitor cell; expanding the progenitor cell under a hypoxic condition; transducing to the progenitor cell a polynucleotide encoding a chimeric receptor; and differentiating the progenitor cell into an immature myeloid cell, wherein the chimeric receptor comprises, from the N-terminus to the C-terminus, an extracellular domain of a receptor to a tumor-associated ligand, a transmembrane domain, a costimulatory domain, and a CD3ζ intracellular domain.

In some embodiments, the immature myeloid cells are administered to a patient having cancer. In some embodiments, the patient having cancer is the same person from whom the progenitor cell is obtained.

Methods for using the prepared compositions to treat cancer are also provided. In some embodiments, the cancer is a solid tumor, a leukemia or lymphoma. In some embodiments, the cancer is a solid tumor. In some embodiments, the cancer is selected from the group consisting of non-small cell lung cancer (NSCLC), small cell lung cancer, prostate cancer, pancreatic ductal carcinoma, neuroblastoma, glioblastoma, ovarian cancer, melanoma, and breast cancer. In some embodiments, the cancer is metastatic.

### BRIEF DESCRIPTION OF THE DRAWINGS

FIG. 1 illustrates a process for preparing an immune cell that expresses a chimeric receptor of the present disclosure.

FIG. 2A-B show the organization of Chimeric CAR-like Immune Receptor (CARIR) as well as an illustrative vector that encodes CARIR and other useful components of an engineered immune cell of the present technology. A. Illustration of CARIR, which comprises an extracellular domain (ED), a CD8 hinge region, a CD8 transmembrane domain, and CD3ζ activation domain. The ED is derived from natural human immune receptor, such as PD-1. B. Illustration of a lentiviral vector encoding CARIR and other useful components, including a protease digestion site P2A, a kill switch (truncated EGFR (tEGFR)). Upon protease treatments, the preprotein can be split into the chimeric receptor and the tEGFR kill switch. LTR: Long Terminal Repeats, EF1α: Elongation Factor 1α promoter, CARIR: CAR-Like Immune Receptor, P2A: Self-cleaving peptide sequences, tEGFR: Truncated Epidermal Growth Factor Receptor, PD-1: extracellular domain of PD-1 receptor, CD3ζ: cytosolic domain of CD3ζ.

FIG. 3A-B show lentiviral mediated CARIR and EGFP kill switch expressions in human monocytic THP-1 cells. THP-1 cells were transduced with lentiviral vector encoding PD-1 CARIR at indicated multiplicity of infection (MOI). A. Offset histograms show CARIR expression, measured by flow cytometry through detecting surface expression of PD-1. B. Offset histograms show the expression of truncated EGFR which serve as a safety kill switch. The cells were gated on live and singlets.

FIG. 4A illustrates additional CAR-like Immune Receptors (CARIR) with extracellular domain derived from the receptors that are relevant in cancer immunotherapy, including SIRPα, Siglec10, CTLA-4, TIM-3, LAG3, CXCR4, CCR2, CXCR2, CCR7, and TREM2.

FIG. 4B-C illustrate the Diagrams for additional CAR-like Immune Receptors (CARIR) with different intracellular domain(s)

FIG. 5A-C show the Lentiviral vector mediated CARIR expression in human monocytic THP-1 cells. A. Dot plots show SIRPα and EGFR surface expression in SIRPα CARIR transduced THP-1 cells. B. Dot plots show Siglec10 and EGFR surface expression in Siglec10 CARIR transduced THP-1 cells. C. Dot plots show TREM2 CARIR expression in transduced THP-1 cells based on the detection of EGFR kill switch. The cells were gated on live and singlets.

FIG. 6 shows Efficient NFκB-1 (p50) gene knockout in human HSCs through CRISPR/Cas9 approach. Human mobilized peripheral blood (MPB) or bone marrow (BM)-derived CD34⁺ hematopoietic stem cells (HSCs) were electroporated with ribonucleoprotein (RNP) complex containing recombinant Cas9 protein as well as guide RNA #1 (gRNA #1, target: TACCCGACCACCATGTCCTT; SEQ ID NO:46) and/or guide RNA #2 (gRNA #2, target: ATATAGATCTGCAACTATGT; SEQ ID NO:47). The % indel among the NFκB-1 (p50) gene was measured by TIDE analysis 6 days later.

FIG. 7A-B show the Engineering of primary human CD34⁺ hematopoietic stem cells (HSCs) to knock out NF-kB1 (p50) and express CARIR. A. Procedure for the same-day human HSCs engineering. B. Offset histograms show CARIR (PD-1) expression on HSCs with or without NF-kB1 (p50) knock out. The cells were gated on live and singlets. NT: no transduction; Mock: mock transduction; CARIR: CARIR transduction only; p50 KO: p50 knock-out only.

FIG. 8A-B show the in vitro expansion of human CD34⁺ hematopoietic stem cells (HSCs). Mobilized peripheral blood (MPB) or bone marrow (BM)-derived human CD34⁺ HSCs were expanded in vitro for a week in culture medium containing TPO, SCF, Flt3L, and UM171. The number of live cells were counted at the indicated time points. A. Fold expansion of MPB-derived CD34⁺ HSCs. The expansion experiment was conducted with cells from 2 different donors, each with 3 independent repeats. The data was shown as mean±SEM. B. Fold expansion of BM-derived CD34⁺ HSCs. The expansion experiment was conducted with cells from one donor in duplicate. Average number of the cells were shown in the figure at each timepoint.

FIG. 9A-G show more myeloid cells differentiation in BM than MPB-derived human CD34⁺ HSCs under either normoxia or hypoxia conditions. Mobilized peripheral blood (MPB) or bone marrow (BM)-derived human CD34⁺ HSCs were cultured in myeloid differentiation medium for 4 days in incubators that maintain either normoxia (N, 20% O2) or hypoxia (H, 1% O2) as indicated. The cells were then analyzed by flow cytometry for the indicated cell surface markers. A-E. Pairwise data comparing under normoxia (N) versus hypoxia (H) culture condition the frequency of cells that were positive for CD11b (A), CD14 (B), CD34 (C), CD38 (D), or CD15 (E). F. Summarized data show the frequency of cells among BM versus MPB-derived CD34⁺ HSCs following myeloid differentiation under normoxia condition. G. Summarized data show the frequency of cells among BM versus MPB-derived CD34⁺ HSCs following myeloid differentiation under hypoxia condition. Cells were gated on live and singlets. Human CD34⁺ HSC samples from 2 BM donors and 3 MPB donors were tested, and the experiment was performed in triplicate. *p<0.05, **p<0.01, ***p<0.001, ****p<0.0001 by Student t test with 2-tail distribution. Ns: not significant.

FIG. 10A-D show more myeloid cells differentiation in BM versus MPB-derived human CD34⁺ HSCs over a time course of 10 days. Mobilized peripheral blood (MPB) or bone marrow (BM)-derived human CD34⁺ HSCs were plated in ultralow attachment plates in myeloid differentiation medium containing M-CSF and GM-CSF. The cells were cultured in the incubator that either maintain normoxia (20% O₂) or hypoxia (1% O₂) as indicated. On day 4, 7, and 10, the cells were analyzed by flow cytometry for cell surface markers CD11b and CD34. A. The percentage of CD11b⁺ myeloid cells differentiated under normoxia condition over the time course. B. The percentage of cells that remain to be CD34⁺ following myeloid differentiated under normoxia condition over the time course. C. The percentage of CD11b⁺ myeloid cells differentiated under hypoxia condition over the time course. D. The percentage of cells that remain to be CD34⁺ following myeloid differentiated under hypoxia condition over the time course. The experiment was conducted in triplicate and 2 to 3 different donors of each sample types were used. Data was presented as mean±SEM. *p<0.05 comparing the BM versus MPB cell sample by student t test with 2-tailed distribution.

FIG. 11A-C show CARIR expression in human monocytic THP-1 cells increases phagocytosis on RM-1^{hPD-L1} target cells. Phagocytosis assay was utilized to evaluate the functionality of CARIR. The CellTrace Violet labeled effector THP-1, CARIR-Δz THP-1 (lacking CD33 signaling domain in the CARIR), or CARIR-z THP-1 cells were pretreated with 1 ng/ml PMA for 24 hours. The effector cells were then co-cultured for 4 hours with CFSE labeled RM-1 or RM-1^{hPD-L1} (RM-1 cells that engineered to overexpress human PD-L1) target cells at the effector to target ratio of 5:1, in the presence or absence of 2 μM cytochalasin D (cyto), 10 μg/ml pembrolizumab biosimilar anti-PD-1 antibody (aPD1), or human IgG4 isotype control (iso). The % phagocytosis was assessed by flow cytometry. A. CARIR transduction efficiency in THP-1 cells as evaluated by flow staining for PD-1. The cells were gated on live, singlets. B. Example flow dot plots showing the % phagocytosis by CARIR-z-THP-1 cells in the presence or absence of RM-1^{hPD-L1} target cells. The cells were gated on live, singlets, and Violet⁺CFSE⁺. C. The bar graph summarizes the % phagocytosis result. The data were expressed as mean±SD. *p<0.05, ****p<0.0001, and ns: not significant, by one-way ANOVA using the Prism software. Multiplicity adjusted p values were reported.

FIG. 12A-D show that CARIR expression in human monocytic THP-1 cells increases their phagocytosis on PD-L1⁺ human triple negative breast cancer cells. To serve as the effectors, human monocytic THP-1 cells were engineered to express either CARIR-Δz (a PD-1 CARIR that lacks CD35 signaling domain) or CARIR-z through lentiviral transduction, and then differentiated to macrophages by PMA treatment. MDA-MB-231 tumor cells that express PD-L1 were used to serve as the target cells. A. A flow chart for the experimental procedures. B. Histogram shows positive PD-L1 expression on MDA-MB-231 cells. C. Representative flow plots showing % phagocytosis on MDA-MB-231 tumor cells. The cells were gated on CellTrace Violet THP-1 macrophages. The CellTrace Violet and CellTrace Yellow double positive population represent macrophages that have phagocytosed target cells. D. Bar graph summarizes the phagocytosis result shown in panel C. ***p<0.001, and ****p<0.0001 by ordinary one-way ANOVA using Prism software.

FIG. 13A-B show that CARIR expression increases the phagocytosis of human THP-1 macrophages on PD-L1⁺ NCI-H358 human lung cancer cells. A. Histogram shows high level of PD-L1 expression in the cultured NCI-H358 tumor cells. B. % phagocytosis on NCI-H358 cells. Human monocytic THP-1 cells were engineered to express either CARIR-Az or CARIR-z through lentiviral transduction, and then differentiated to macrophages by PMA pretreatment. CellTrace Violet labeled THP-1 effectors were co-cultured with CellTrace Yellow labeled NCI-H358 tumor cells for 4 hours, followed by flow cytometry analysis of the % phagocytosis. Cells were gated on live, singlets, and violet+ cells. The events that were double positive for CellTrace Violet and CellTrace Yellow were considered as phagocytic events. Data was presented as mean±SD. *p<0.05, and **p<0.01 by ordinary one-way ANOVA using Prism software.

FIG. 14A-D show that Infusion of engineered myeloid cells slows 4T1 tumor growth and prolongs survival in syngeneic mouse model. A. Schematic timeline for the animal experiment. Syngeneic Balb/c mice were subcutaneously implanted with 5×10⁴ 4T1 breast cancer cells on day-7. Starting on day 0, the mice were treated with 3 weekly dose of 10×10⁶ engineered mouse immature myeloid cells expressing murine analog of CARIR (CARIR-IMC) or with NFκB-1 (p50) knocked out (p50^{-/-}-IMC) or PBS as indicated. B. Tumor volume measurements. C. Probability of survival. D. Body weight over the course of the experiment. Data are presented as mean±SEM. *p<0.01: CARIR-IMC vs WT-IMC or PBS, p50^{-/-}-IMC vs WT-IMC or PBS, One-way ANOVA. #p<0.05: CARIR-IMC vs PBS,p<0.01: CARIR-IMC vs WT-IMC, p50^{-/-}-IMC vs WT-IMC or PBS, by Gehan-Breslow-Wilcoxon test using prism software.

FIG. 15 illustrates the process for manufacturing engineered myeloid cells for adoptive cell therapy.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

### Preparation of Immune Cells Expressing a Chimeric Receptor

The instant disclosure provides compositions and methods for efficient production of immune cells suitable for treating cancer. The efficacy of current immune cell therapies for treating solid cancers is limited by several factors. First, there is inherent heterogeneity among cancer cells. Second, myeloid-derived suppressor cells (MDSC) and/or tumor promoting M2 macrophages lead to a strong immunosuppressive tumor microenvironment (TME). Third, the conventional adoptive transferred immune cells are often inefficient on solid tumor penetration.

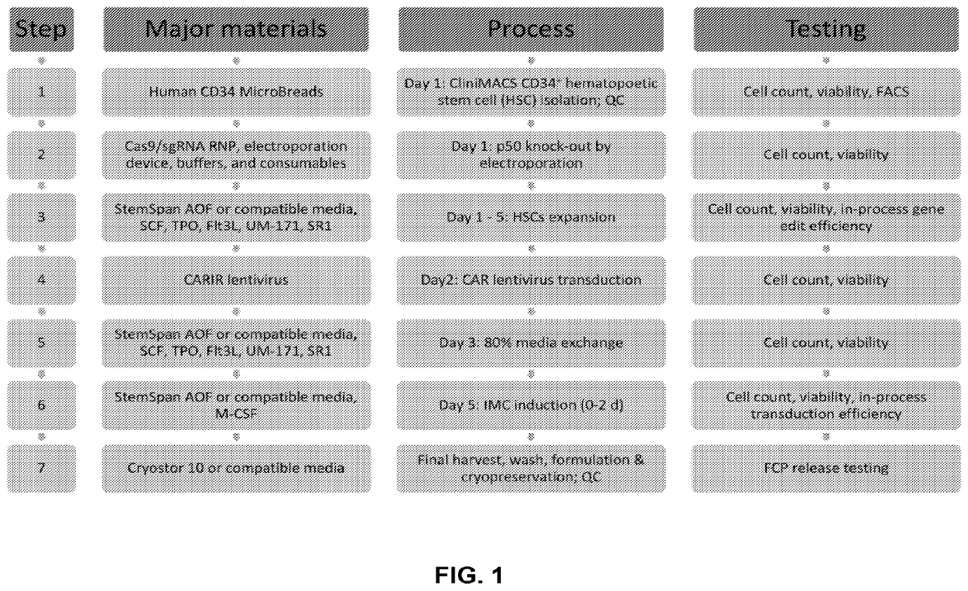

Such challenges, however, can be overcome by the engineered immune cells prepared by the instantly disclosed methods. In an example embodiment and with reference to FIG. 1, the preparation method entails:
(a) acquiring a human autologous bone marrow-derived CD34⁺ hematopoietic stem cell (HSC);
(b) inactivating the NFκB1 (p50) gene in the HSC;
(c) expanding the HSC under hypoxic conditions;
(d) introducing to the HSC a polynucleotide that encodes a chimeric receptor (CARIR) of the instant disclosure; and
(e) differentiating the HSC into an immature myeloid cell (IMC).

It is important to note that not every step is required in the method, and the order of these steps can be adjusted. For instance, the genetic manipulation steps (e.g., p50 inactivation and transduction) can be prior to or after the HSC-to-IMC differentiation. Also, the hypoxic expansion can be carried out for both the HSC and the IMC, either of them, or neither of them.

In one embodiment, the order is (a) progenitor cell acquisition=>(b) p50 inactivation=>(c) cell expansion=>(d) chimeric receptor transduction=>(e) cell differentiation. In another embodiment, the order is (a)=>(b)=>(d)=>(c)=>(e). In another embodiment, the order is (a)=>(d)=>(b)=>(c)=>(e). In another embodiment, the order is (a)=>(c)=>(b)=>(d)=>(e). In another embodiment, the order is (a)=>(c)=>(d)=>(b)=>(e). In another embodiment, the order is (a)=>(e)=>(b)=>(d)=>(c). In another embodiment, the order is (a)=>(e)=>(d)=>(b)=>(c). In another embodiment, the order is (b)=>(a)=>(c)=>(d)=>(e).

Each of these steps is described in more detail below.

### Chimeric Receptor and Cell Transduction

In various aspects of the present technology, a progenitor cell or an immune cell is transduced to express a chimeric receptor that helps target the immune cell to a tumor cell. The chimeric receptor, also referred to as a “CAR-like immune receptor,” or “CARIR”, like a conventional CAR, includes an extracellular targeting domain, a transmembrane domain, and one or more costimulatory domains or signal domains.

A conventional CAR includes an antibody or antigen-binding fragment, such as a single chain fragment (scFv), as the extracellular targeting domain to bind to a target molecule, such as a tumor-associated antigen (TAA). By contrast, the chimeric receptors of the present disclosure, in some embodiments, employs the extracellular binding domain of a natural receptor protein that can bind to the target protein, through a conventional ligand-receptor interaction.

For instance, various inhibitory receptors are expressed on immune cells, such as myeloid cells. Non-limiting examples include PD1 which can bind to ligand PD-L1, SIRPα which can bind to CD47, Siglec-10 which can bind to CD52 and CD24, CTLA-4 which can bind to B7-1 and B7-2, TIM-3 which can bind to Gal-9, PtdSer, HMGB1 and CEACAM1, and LAG3 which can bind to MHC class II and FGL1. In some embodiments, the receptor is PD1, SIRPα, Siglec-10, or CTLA-4.

Various chemokine receptors which are expressed on immune cells also include such extracellular domains capable of binding to the corresponding ligands. For instance, the chemokine receptor CXCR4 can bind to SDF-1, ubiquitin and MIF, CCR2 can bind to chemokine CCL2, CXCR2 can bind to CXCL1, and CCR7 can bind to CCL19 and CCL21.

As demonstrated in the accompanying experimental examples, when a CARIR that contained the extracellular domain of PD-1 was expressed on an immune cell, such as an immature myeloid cell or a macrophage, the engineered immune cell was able to bind to tumor cells expressing PD-L1 and initiate phagocytosis (Example 5), and adoptive transfer CARIR-expressing immature myeloid cells leading to inhibition of tumor growth (Example 6).

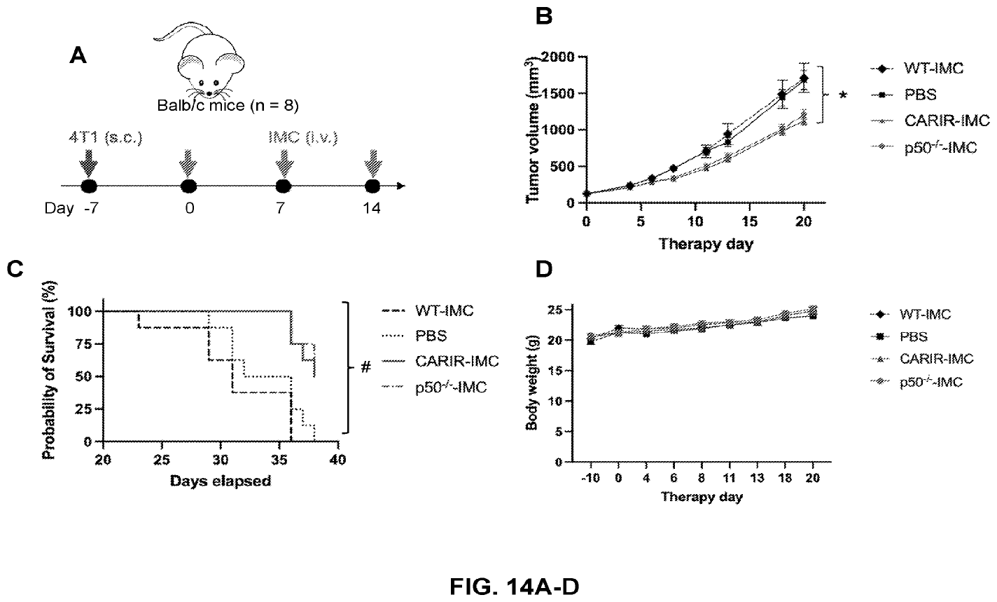

Such anti-tumor effects of the CARIR molecules, however, were unexpected. It is commonly known that therapeutic antibodies typically have a binding affinity on the scale of 0.1-10 nM (EC₅₀). For instance, the EC₅₀ of anti-PD1 antibodies pembrolizumab and nivolumab are 2.440 nM and 5.697 nM, respectively. The affinity between the natural ligands and receptors, however, can be considerably lower. For example, the EC₅₀ between PD-1 and PD-L1 is 7 μM and that between SIRPα and CD47 is 2 μM, both of which are about 1000 times weaker than antibodies. With a 1000-fold lower binding affinity but achieving in vivo anti-tumor effects in a model which is known for its poor response to traditional anti-PD1/PD-L1 antibody blockade therapy (FIG. 14B-C), the instantly disclosed CARIR truly have exhibited unexpected results.

The full-length sequences of these example receptors are provided in Table 1, along with their extracellular targeting domains. For each receptor, a core extracellular domain (ECD) is provided, which shows the minimum sequence required for binding to the ligand. Sometimes, an extended ECD sequence is also provided, which is slightly longer than the core ECD sequence.

TABLE 1
Extracellular Domains of Example Receptors

| Receptor | Sequences |
|---|---|
| PD1 | Full length: (SEQ ID NO: 1) |
| | MQIPQAPWPVVWAVLQLGWRPGWFLDSPDRPWNPPTFSPALLVVTEGDNATFTCSFSN |
| | TSESFVLNWYRMSPSNQTDKLAAFPEDRSQPGQDCRFRVTQLPNGRDFHMSVVRARRN |
| | DSGTYLCGAISLAPKAQIKESLRAELRVTERRAEVPTAHPSPSPRPAGQFQTLVVGVV |
| | GGLLGSLVLLVWVLAVICSRAARGTIGARRTGQPLKEDPSAVPVFSVDYGELDFQWRE |
| | KTPEPPVPCVPEQTEYATIVFPSGMGTSSPARRGSADGPRSAQPLRPEDGHCSWPL |
| | Core ECD binding sequence: (SEQ ID NO: 2) |
| | MSPSNQTDKLAAFPEDRSQPGQDCRFRVTQ |
| | Extended ECD binding sequence: (SEQ ID NO: 3) |
| | MSPSNQTDKLAAFPEDRSQPGQDCRFRVTQLPNGRDFHMSVVRARRNDSGTYLCGAIS |
| | LAPKAQIKESLRAELRVTERRAEVPTAHPSPSPRPAGQFQTLV |
| SIRPα | Full length: (SEQ ID NO: 4) |
| | MEPAGPAPGRLGPLLCLLLAASCAWSGVAGEEELQVIQPDKSVLVAAGETATLRCTAT |
| | SLIPVGPIQWFRGAGPGRELIYNQKEGHFPRVTTVSDLTKRNNMDFSIRIGNITPADA |
| | GTYYCVKFRKGSPDDVEFKSGAGTELSVRAKPSAPVVSGPAARATPQHTVSFTCESHG |
| | FSPRDITLKWFKNGNELSDFQTNVDPVGESVSYSIHSTAKVVLTREDVHSQVICEVAH |
| | VTLQGDPLRGTANLSETIRVPPTLEVTQQPVRAENQVNVTCQVRKFYPQRLQLTWLEN |
| | GNVSRTETASTVTENKDGTYNWMSWLLVNVSAHRDDVKLTCQVEHDGQPAVSKSHDLK |
| | VSAHPKEQGSNTAAENTGSNERNIYIVVGVVCTLLVALLMAALYLVRIRQKKAQGSTS |
| | STRLHEPEKNAREITQDTNDITYADLNLPKGKKPAPQAAEPNNHTEYASIQTSPQPAS |
| | EDTLTYADLDMVHLNRTPKQPAPKPEPSFSEYASVQVPRK |
| | Core ECD binding sequence: (SEQ ID NO: 5) |
| | EEELQVIQPDKSVLVAAGETATLRCTATSLIPVGPIQWFRGAGPGRELIYNQKEGHFP |
| | RVTTVSDLTKRNNMDFSIRIGNITPADAGTYYCVKFRKGSPDDVE |
| | Extended ECD binding sequence: (SEQ ID NO: 6) |
| | EEELQVIQPDKSVLVAAGETATLRCTATSLIPVGPIQWFRGAGPGRELIYNQKEGHFP |
| | RVTTVSDLTKRNNMDFSIRIGNITPADAGTYYCVKFRKGSPDDVEFKSGAGTELSVRA |
| | KPSAPVVSGPAARATPQHTVSFTCESHGFSPRDITLKWFKNGNELSDFQTNVDPVGES |
| | VSYSIHSTAKVVLTREDVHSQVICEVAHVTLQGDPLRGTANLSETIRVPPTLEVTQQP |
| | VRA |
| Siglec-10 | Full length: (SEQ ID NO: 7) |
| | MLLPLLLSSLLGGSQAMDGRFWIRVQESVMVPEGLCISVPCSFSYPRQDWTGSTPAYG |
| | YWFKAVTETTKGAPVATNHQSREVEMSTRGRFQLTGDPAKGNCSLVIRDAQMQDESQY |
| | FFRVERGSYVRYNFMNDGFFLKVTALTQKPDVYIPETLEPGQPVTVICVFNWAFEECP |
| | PPSFSWTGAALSSQGTKPTTSHFSVLSFTPRPQDHNTDLTCHVDFSRKGVSAQRTVRL |
| | RVAYAPRDLVISISRDNTPALEPQPQGNVPYLEAQKGQFLRLLCAADSQPPATLSWVL |
| | QNRVLSSSHPWGPRPLGLELPGVKAGDSGRYTCRAENRLGSQQRALDLSVQYPPENLR |
| | VMVSQANRTVLENLGNGTSLPVLEGQSLCLVCVTHSSPPARLSWTQRGQVLSPSQPSD |
| | PGVLELPRVQVEHEGEFTCHARHPLGSQHVSLSLSVHYSPKLLGPSCSWEAEGLHCSC |
| | SSQASPAPSLRWWLGEELLEGNSSQDSFEVTPSSAGPWANSSLSLHGGLSSGLRLRCE |
| | AWNVHGAQSGSILQLPDKKGLISTAFSNGAFLGIGITALLFLCLALIIMKILPKRRTQ |
| | TETPRPRFSRHSTILDYINVVPTAGPLAQKRNQKATPNSPRTPLPPGAPSPESKKNQK |
| | KQYQLPSFPEPKSSTQAPESQESQEELHYATLNFPGVRPRPEARMPKGTQADYAEVKF |
| | Q |
| | Core ECD binding sequence: (SEQ ID NO: 8) |
| | MDGRFWIRVQESVMVPEGLCISVPCSFSYPRQDWTGSTPAYGYWFKAVTETTKGAPVA |
| | TNHQSREVEMSTRGRFQLTGDPAKGNCSLVIRDAQMQDESQYFFRVERGSYVRYNEMN |
| | DGFFLKVTALTQK |
| | Extended ECD binding sequence: (SEQ ID NO: 9) |
| | MDGRFWIRVQESVMVPEGLCISVPCSFSYPRQDWTGSTPAYGYWFKAVTETTKGAPVA |
| | TNHQSREVEMSTRGRFQLTGDPAKGNCSLVIRDAQMQDESQYFFRVERGSYVRYNEMN |
| | DGFFLKVTALTQKPDVYIPETLEPGQPVTVICVENWAFEECPPPSFSWTGAALSSQGT |
| | KPTTSHFSVLSFTPRPQDHNTDLTCHVDFSRKGVSAQRTVRLRVAYAPRDLVISISRD |
| | NTPALE |
| CTLA-4 | Full length: (SEQ ID NO: 10) |
| | MACLGFQRHKAQLNLATRTWPCTLLFFLLFIPVFCKAMHVAQPAVVLASSRGIASFVC |
| | EYASPGKATEVRVTVLRQADSQVTEVCAATYMMGNELTFLDDSICTGTSSGNQVNLTI |
| | QGLRAMDTGLYICKVELMYPPPYYLGIGNGTQIYVIDPEPCPDSDELLWILAAVSSGL |
| | FFYSFLLTAVSLSKMLKKRSPLTTGVYVKMPPTEPECEKQFQPYFIPIN |
| | Core ECD binding sequence: (SEQ ID NO: 11) |
| | RGIASFVCEYASPGKATEVRVTVLRQADSQVTEVCAATYMMGNELTFLDDSICTGTSS |
| | GNQVNLTIQGLRAMDTGLYICKVELMYPPPYYLGIGNGTQI |
| TIM-3 | Full length: (SEQ ID NO: 12) |
| (havcr2) | MFSHLPFDCVLLLLLLLLTRSSEVEYRAEVGQNAYLPCFYTPAAPGNLVPVCWGKGAC |
| | PVFECGNVVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGSLPDINLT |
| | QISTLANELRDSRLANDLRDSGATIRIGIYIGAGICAGLALALIFGALIFKWYSHSKE |
| | KIQNLSLISLANLPPSGLANAVAEGIRSEENIYTIEENVYEVEEPNEYYCYVSSRQQP |
| | SQPLGCRFAMP |
| | Core ECD binding sequence: (SEQ ID NO: 13) |
| | EVEYRAEVGQNAYLPCFYTPAAPGNLVPVCWGKGACPVFECGNVVLRTDERDVNYWTS |
| | RYWLNGDFRKGDVSLTIENVTLADSGIYCCRIQIPGIMNDEKFNLKLVIKPAKVTPAP |
| | TRQRDFTAAFPRMLTTRGHGPAETQTLGSLPDINLTQISTLANELRDSRLANDLRDSG |
| | ATIRIG |
| LAG3 | Full length: (SEQ ID NO:14) |
| | MWEAQFLGLLFLQPLWVAPVKPLQPGAEVPVVWAQEGAPAQLPCSPTIPLQDLSLLRR |
| | AGVTWQHQPDSGPPAAAPGHPLAPGPHPAAPSSWGPRPRRYTVLSVGPGGLRSGRLPL |
| | QPRVQLDERGRQRGDFSLWLRPARRADAGEYRAAVHLRDRALSCRLRLRLGQASMTAS |
| | PPGSLRASDWVILNCSFSRPDRPASVHWFRNRGQGRVPVRESPHHHLAESFLFLPQVS |
| | PMDSGPWGCILTYRDGFNVSIMYNLTVLGLEPPTPLTVYAGAGSRVGLPCRLPAGVGT |
| | RSFLTAKWTPPGGGPDLLVTGDNGDFTLRLEDVSQAQAGTYTCHIHLQEQQLNATVTL |
| | AIITVTPKSFGSPGSLGKLLCEVTPVSGQERFVWSSLDTPSQRSFSGPWLEAQEAQLL |
| | SQPWQCQLYQGERLLGAAVYFTELSSPGAQRSGRAPGALPAGHLLLFLILGVLSLLLL |
| | VTGAFGFHLWRRQWRPRRFSALEQGIHPPQAQSKIEELEQEPEPEPEPEPEPEPEPEP |
| | EQL |
| | Core ECD binding sequence: (SEQ ID NO: 15) |
| | LQPGAEVPVVWAQEGAPAQLPCSPTIPLQDLSLLRRAGVTWQHQPDSGPPAAAPGHPL |
| | APGPHPAAPSSWGPRPRRYTVLSVGPGGLRSGRLPLQPRVQLDERGRQRGDFSLWLRP |
| | ARRADAGEYRAAVHLRDRALSCRLRLRLGQASMTASPPGSLRASDWVILNCSFSRPDR |
| | PASVHWFRNRGQGRVPVRESPHHHLAESFLFLPQVSPMDSGPWGCILTYRDGFNVSIM |
| | YNLTV |
| | Extended ECD binding sequence: (SEQ ID NO: 16) |
| | LQPGAEVPVVWAQEGAPAQLPCSPTIPLQDLSLLRRAGVTWQHQPDSGPPAAAPGHPL |
| | APGPHPAAPSSWGPRPRRYTVLSVGPGGLRSGRLPLQPRVQLDERGRQRGDFSLWLRP |
| | ARRADAGEYRAAVHLRDRALSCRLRLRLGQASMTASPPGSLRASDWVILNCSFSRPDR |
| | PASVHWFRNRGQGRVPVRESPHHHLAESFLFLPQVSPMDSGPWGCILTYRDGENVSIM |
| | YNLTVLGLEPPTPLTVYAGAGSRVGLPCRLPAGVGTRSFLTAKWTPPGGGPDLLVTGD |
| | NGDFTLRLEDVSQAQAGTYTCHIHLQEQQLNATVTLAIITVTPKSFGSPGSLGKLLCE |
| | VTPVS |
| CXCR4 | Full length: (SEQ ID NO: 17) |
| | MEGISIYTSDNYTEEMGSGDYDSMKEPCFREENANFNKIFLPTIYSIIFLTGIVGNGL |
| | VILVMGYQKKLRSMTDKYRLHLSVADLLFVITLPFWAVDAVANWYFGNFLCKAVHVIY |
| | TVNLYSSVLILAFISLDRYLAIVHATNSQRPRKLLAEKVVYVGVWIPALLLTIPDFIF |
| | ANVSEADDRYICDRFYPNDLWVVVFQFQHIMVGLILPGIVILSCYCIIISKLSHSKGH |
| | QKRKALKTTVILILAFFACWLPYYIGISIDSFILLEIIKQGCEFENTVHKWISITEAL |
| | AFFHCCLNPILYAFLGAKFKTSAQHALTSVSRGSSLKILSKGKRGGHSSVSTESESSS |
| | FHSS |
| | Core ECD binding sequence: (SEQ ID NO: 18) |
| | MEGISIYTSDNYTEEMGSGDYDSMKEPCFREENANENK |
| CCR2 | Full length: (SEQ ID NO: 19) |
| | MLSTSRSRFIRNTNESGEEVTTFFDYDYGAPCHKEDVKQIGAQLLPPLYSLVFIFGFV |
| | GNMLVVLILINCKKLKCLTDIYLLNLAISDLLFLITLPLWAHSAANEWVFGNAMCKLF |
| | TGLYHIGYFGGIFFIILLTIDRYLAIVHAVFALKARTVTFGVVTSVITWLVAVFASVP |
| | GIIFTKCQKEDSVYVCGPYFPRGWNNFHTIMRNILGLVLPLLIMVICYSGILKTLLRC |
| | RNEKKRHRAVRVIFTIMIVYFLFWTPYNIVILLNTFQEFFGLSNCESTSQLDQATQVT |
| | ETLGMTHCCINPIIYAFVGEKFRRYLSVFFRKHITKRFCKQCPVFYRETVDGVTSTNT |
| | PSTGEQEVSAGL |
| | Core ECD binding sequence: (SEQ ID NO: 20) |
| | LSTSRSRFIRNTNESGEEVTTFFDYDYGAPCHKFDVK |
| CXCR2 | Full length: (SEQ ID NO:21) |
| | MEDFNMESDSFEDFWKGEDLSNYSYSSTLPPFLLDAAPCEPESLEINKYFVVIIYALV |
| | FLLSLLGNSLVMLVILYSRVGRSVTDVYLLNLALADLLFALTLPIWAASKVNGWIFGT |
| | FLCKVVSLLKEVNFYSGILLLACISVDRYLAIVHATRTLTQKRYLVKFICLSIWGLSL |
| | LLALPVLLFRRTVYSSNVSPACYEDMGNNTANWRMLLRILPQSFGFIVPLLIMLFCYG |
| | FTLRTLFKAHMGQKHRAMRVIFAVVLIFLLCWLPYNLVLLADTLMRTQVIQETCERRN |
| | HIDRALDATEILGILHSCLNPLIYAFIGQKFRHGLLKILAIHGLISKDSLPKDSRPSF |
| | VGSSSGHTSTTL |
| | Core ECD binding sequence: (SEQ ID NO: 22) |
| | EDFNMESDSFEDFWKGEDLSNYSYSSTLPPFLLDAAPCEPESLEINK |
| CCR7 | Full length: (SEQ ID NO: 23) |
| | MDLGKPMKSVLVVALLVIFQVCLCQDEVIDDYIGDNTTVDYTLFESLCSKKDVRNFKA |
| | WFLPIMYSIICFVGLLGNGLVVLTYIYFKRLKTMTDTYLLNLAVADILFLLTLPFWAY |
| | SAAKSWVFGVHFCKLIFAIYKMSFFSGMLLLLCISIDRYVAIVQAVSAHRHRARVLLI |
| | SKLSCVGIWILATVLSIPELLYSDLQRSSSEQAMRCSLITEHVEAFITIQVAQMVIGE |
| | LVPLLAMSFCYLVIIRTLLQARNFERNKAIKVIIAVVVVFIVFQLPYNGVVLAQTVAN |
| | FNITSSTCELSKQLNIAYDVTYSLACVRCCVNPFLYAFIGVKFRNDLFKLFKDLGCLS |
| | QEQLRQWSSCRHIRRSSMSVEAETTTTFSP |
| | Core ECD binding sequence: (SEQ ID NO: 24) |
| | QDEVIDDYIGDNTTVDYTLFESLCSKKDVRNFKA |
| CD80 | Full length: (SEQ ID NO: 41) |
| | MGHTRRQGTSPSKCPYLNFFQLLVLAGLSHFCSGVIHVTKEVKEVATLSCGHNVSVEE |
| | LAQTRIYWQKEKKMVLTMMSGDMNIWPEYKNRTIFDITNNLSIVILALRPSDEGTYEC |
| | VVLKYEKDAFKREHLAEVTLSVKADFPTPSISDFEIPTSNIRRIICSTSGGFPEPHLS |
| | WLENGEELNAINTTVSQDPETELYAVSSKLDFNMTTNHSFMCLIKYGHLRVNQTENWN |
| | TTKQEHFPDNLLPSWAITLISVNGIFVICCLTYCFAPRCRERRRNERLRRESVRPV |
| | Core ECD binding sequence: (SEQ ID NO: 42) |
| | IFDITNNLSIVIL |
| | Extended ECD binding sequence: (SEQ ID NO: 43) |
| | VIHVTKEVKEVATLSCGHNVSVEELAQTRIYWQKEKKMVLTMMSGDMNIWPEYKNRTI |
| | FDITNNLSIVILALRPSDEGTYECVVLKYEKDAFKREHLAEVTLSVK |

Accordingly, in some embodiments, a chimeric receptor is provided which includes, from the N-terminus to the C-terminus, an extracellular domain of a receptor to a tumor-associated ligand, a transmembrane domain, a costimulatory domain, and a CD3ζ intracellular domain. In some embodiments, the extracellular domain includes a ligand-binding domain.

Example receptors include PD1, SIRPα, Siglec-10, CTLA-4, CXCR-4, CCR-2, CXCR2, CCR7, CD80, TIM-3, LAG3 and TREM2. Example ligand-binding domains include the sequences provided in SEQ ID NO:2, 5, 8, 11, 13, 15, 18, 20, 22, 24, 42 and 43. Additional examples include 3, 6, 9, and 16.

Still, further examples include those that are biologically equivalent to those exemplified above. A biological equivalent to an extracellular binding domain is one that has at least 75%, 80%, 85%, 90%, 95%, 98%, or 99% sequence identity to a reference extracellular binding domain, such as those provided in Table 1. In some embodiments, the substitutions allowed in the designated sequence identities are conservative amino acid substitutions.

The extracellular targeting domain can target the engineered immune cell, which expresses the extracellular targeting domain, to a tumor tissue where the corresponding ligand is expressed. In addition to the extracellular domain, the chimeric receptor also includes other useful elements.

In some embodiments, the chimeric receptor further includes a transmembrane (TM) domain. A transmembrane domain can be designed to be fused to the extracellular domain, optionally through a hinge domain. It can similarly be fused to an intracellular domain, such as a costimulatory domain. In some embodiments, the transmembrane domain can include the natural transmembrane region of a costimulatory domain (e.g., the TM region of a CD28T or 4-IBB employed as a costimulatory domain) or the natural transmembrane domain of a hinge region (e.g., the TM region of a CD8 alpha or CD28T employed as a hinge domain). Example sequences are provided in Table 2.

In some embodiments, the transmembrane domain can be selected or modified by amino acid substitution to avoid binding of such domains to the transmembrane domains of the same or different surface membrane proteins to minimize interactions with other members of the receptor complex. A transmembrane domain can be derived either from a natural or from a synthetic source. When the transmembrane domain is derived from a naturally-occurring source, the domain can be derived from any membrane-bound or transmembrane protein. In some embodiments, a transmembrane domain is derived from CD2, CD3 delta, CD3 epsilon, CD3 gamma, CD4, CD7, CD8a, CD8, CD11a (ITGAL), CD 1 ib (ITGAM), CD11c (ITGAX), CD11d (ITGAD), CD18 (ITGB2), CD19 (B4), CD27 (TNFRSF7), CD28, CD28T, CD29 (ITGB 1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF 1), CD158A (KTR2DL1), CD158B1 (KIR2DL2), CD158B2 (KIR2DL3), CD158C (KTR3DP1), CD158D (KTRDL4), CD158F 1 (KTR2DL5A), CD158F2 (KTR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (T FRSF18), inducible T cell co-stimulator (ICOS), LFA-1 (CD1 1a/CD18), KG2C, DAP-10, ICAM-1, Kp80 (KLRF 1), IL-2R beta, IL-2R gamma, IL-7R alpha, LFA-1, SLAMF9, LAT, GADS (GrpL), SLP-76 (LCP2), PAGI/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and combinations thereof.

In some embodiments, the transmembrane domain can include a sequence that spans a cell membrane, but extends into the cytoplasm of a cell and/or into the extracellular space. For example, a transmembrane can include a membrane-spanning sequence which itself can further include 1, 2, 3, 4, 5, 6, 7, 8, 9, 10 or more amino acids that extend into the cytoplasm of a cell, and/or the extracellular space. Thus, a transmembrane domain includes a membrane-spanning region, yet can further comprise an amino acid(s) that extend beyond the internal or external surface of the membrane itself; such sequences can still be considered to be a “transmembrane domain.”

In some embodiments, the transmembrane domain of a chimeric receptor of the instant disclosure includes the human CD8α transmembrane domain (SEQ ID NO:28). In some embodiments, the CD8α transmembrane domain is fused to the extracellular domain through a hinge region. In some embodiments, the hinge region includes the human CD8α hinge (SEQ ID NO:27).

In some embodiments, the transmembrane domain is fused to the cytoplasmic domain through a short linker. Optionally, the short peptide or polypeptide linker, preferably between 2 and 10 amino acids in length can form the linkage between the transmembrane domain and a proximal cytoplasmic signaling domain of the chimeric receptor. A glycine-serine doublet (GS), glycine-serine-glycine triplet (GSG), or alanine-alanine-alanine triplet (AAA) provides a suitable linker.

In some embodiments, the chimeric receptor further includes a costimulatory domain. In some embodiments, the costimulatory domain is positioned between the transmembrane domain and an activating domain. Example costimulatory domains include, but are not limited to, CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD18 (ITGB2), CD19 (B4), CD27 (T FRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD 100 (SEMA4D), CD 103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KTR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (TNFRSF 18), inducible T cell co-stimulator (ICOS), LFA-1 (CD 11a/CD 18), KG2C, DAP-10, ICAM-1, Kp80 (KLRF1), IL-2R beta, IL-2R gamma, IL-7R alpha, LFA-1, SLAMF9, LAT, GADS (GrpL), SLP-76 (LCP2), PAGI/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

In some embodiments, the costimulatory domain is selected from the group consisting of CD80, CD86, CD40, 41BB, OX40, and CD28. Some example sequences are provided is Table 2.

In some embodiments, the cytoplasmic portion of the chimeric receptor also includes a signaling/activation domain. In one embodiment, the signaling/activation domain is the CD3ζ domain (SEQ ID NO:39), or is an amino acid sequence having at least about 80%, 85%, 90%, 95%, 98% or 99% sequence identity to the CD3ζ domain.

In some embodiments, the chimeric receptor also includes a leader peptide (also referred to herein as a “signal peptide” or “signal sequence”). The inclusion of a signal sequence in a chimeric receptor is optional. If a leader sequence is included, it can be expressed on the N terminus of the chimeric receptor. Such a leader sequence can be synthesized, or it can be derived from a naturally occurring molecule. An example leader peptide is the human CSF-2 signal peptide (SEQ ID NO:26).

In some embodiments, the chimeric receptor of the present disclosure includes a leader peptide (P), an extracellular targeting domain (T), a hinge domain (H), a transmembrane domain (T), one or more costimulatory regions (C), and an activation domain (A), wherein the chimeric receptor is configured according to the following: P-T-H-T-C-A. In some embodiments the components of the chimeric receptor are optionally joined though a linker sequence, such as AAA or GSG. Some example sequences are provided in Table 2.

TABLE 2
Representative Elements of the Chimeric Receptor

| Elements | Sequences |
|---|---|
| human EF1α | GGCTCCGGTGCCCGTCAGTGGGCAGAGCGCACATCGCCCACAGTCCCCGAGAAGITGG |
| promoter (SEQ | GGGGAGGGGTCGGCAATTGAACCGGTGCCTAGAGAAGGTGGCGCGGGGTAAACTGGGA |
| ID NO: 25) | AAGTGATGTCGTGTACTGGCTCCGCCTTTTTCCCGAGGGTGGGGGAGAACCGTATATA |
| | AGTGCAGTAGTCGCCGTGAACGTTCTTTTTCGCAACGGGTTTGCCGCCAGAACACAGG |
| | TAAGTGCCGTGTGTGGTTCCCGCGGGCCTGGCCTCTTTACGGGTTATGGCCCTTGCGT |
| | GCCTTGAATTACTTCCACCTGGCTGCAGTACGTGATTCTTGATCCCGAGCTTCGGGTT |
| | GGAAGTGGGTGGGAGAGTTCGAGGCCTTGCGCTTAAGGAGCCCCTTCGCCTCGTGCTT |
| | GAGTTGAGGCCTGGCCTGGGCGCTGGGGCCGCCGCGTGCGAATCTGGTGGCACCTTCG |
| | CGCCTGTCTCGCTGCTTTCGATAAGTCTCTAGCCATTTAAAATTTTTGATGACCTGCT |
| | GCGACGCTTTTTTTCTGGCAAGATAGTCTTGTAAATGCGGGCCAAGATCTGCACACTG |
| | GTATTTCGGTTTTTGGGGCCGCGGGCGGCGACGGGGCCCGTGCGTCCCAGCGCACATG |
| | TTCGGCGAGGCGGGGCCTGCGAGCGCGGCCACCGAGAATCGGACGGGGGTAGTCTCAA |
| | GCTGGCCGGCCTGCTCTGGTGCCTGGTCTCGCGCCGCCGTGTATCGCCCCGCCCTGGG |
| | CGGCAAGGCTGGCCCGGTCGGCACCAGTTGCGTGAGCGGAAAGATGGCCGCTTCCCGG |
| | CCCTGCTGCAGGGAGCTCAAAATGGAGGACGCGGCGCTCGGGAGAGCGGGCGGGTGAG |
| | TCACCCACACAAAGGAAAAGGGCCTTTCCGTCCTCAGCCGTCGCTTCATGTGACTCCA |
| | CGGAGTACCGGGCGCCGTCCAGGCACCTCGATTAGTTCTCGAGCTTTTGGAGTACGTC |
| | GTCTTTAGGTTGGGGGGAGGGGTTTTATGCGATGGAGTTTCCCCACACTGAGTGGGTG |
| | GAGACTGAAGTTAGGCCAGCTTGGCACTTGATGTAATTCTCCTTGGAATTTGCCCTTT |
| | TTGAGTTTGGATCTTGGTTCATTCTCAAGCCTCAGACAGTGGTTCAAAGTTTTTTTCT |
| | TCCATTTCAGGTGTCGTGA |
| human CSF-2 | MWLQSLLLLGTVACSIS |
| signal peptide | |
| (SEQ ID NO: 26) | |
| human CD8α | KPTTTPAPRPPTPAPTIASQPLSLRPEACRPAAGGAVHTRGLDFACDIY |
| hinge (SEQ ID | |
| NO: 27) | |
| human CD8α | IWAPLAGTCGVLLLSLVITLY |
| transmembrane | |
| domain (SEQ ID | |
| NO: 28) | |
| P2A protease | ATNFSLLKQAGDVEENPGP |
| site (SEQ ID | |
| NO: 29) | |
| T2A protease | EGRGSLLTCGDVEENPGP |
| site (SEQ ID | |
| NO: 30) | |
| human CD40 | KKVAKKPTNKAPHPKQEPQEINFPDDLPGSNTAAPVQETLHGCQPVTQEDGKESRISV |
| isoform 1 | QERQ |
| cytoplasmic | |
| domain (SEQ ID | |
| NO: 31) | |
| human IL-12 | Coding sequence (SEQ ID NO: 32) |
| fusion (codon | ATGTGCCACCAGCAACTTGTAATAAGTTGGTTCTCACTGGTCTTCCTTGCGAGCCCGT |
| optimized) | TGGTAGCAATCTGGGAACTGAAAAAGGACGTATATGTGGTGGAGCTCGATTGGTATCC |
| | CGACGCACCCGGGGAGATGGTTGTCCTCACCTGTGATACCCCAGAGGAAGACGGTATC |
| | ACTTGGACCTTGGATCAGTCCTCCGAGGTGCTTGGAAGCGGAAAAACTCTGACGATAC |
| | AGGTGAAAGAATTCGGCGACGCGGGACAGTATACATGCCACAAAGGCGGGGAGGTACT |
| | GTCACATTCACTCCTGCTCTTGCATAAAAAGGAGGACGGGATCTGGAGCACTGATATT |
| | TTGAAAGACCAAAAAGAACCAAAAAACAAGACCTTCCTCAGGTGTGAGGCCAAGAATT |
| | ATAGTGGACGCTTCACCTGCTGGTGGCTGACCACAATTAGTACTGACTTGACTTTCTC |
| | AGTAAAGAGTTCCAGGGGGAGTAGTGACCCCCAAGGGGTAACCTGCGGGGCCGCGACT |
| | TTGTCAGCGGAACGGGTGCGAGGGGATAATAAGGAATATGAGTATTCAGTGGAGTGCC |
| | AGGAAGACTCTGCATGTCCCGCAGCAGAGGAAAGCCTGCCGATAGAGGTAATGGTTGA |
| | CGCCGTCCATAAGCTCAAATACGAGAACTACACAAGCTCTTTCTTCATACGGGACATT |
| | ATTAAGCCAGACCCCCCGAAGAATCTTCAACTTAAACCGTTGAAAAATAGTCGACAAG |
| | TGGAAGTCAGTTGGGAATATCCAGACACCTGGAGTACGCCACACAGCTATTTTTCCTT |
| | GACATTTTGCGTCCAGGTTCAGGGGAAATCAAAGCGCGAGAAGAAGGATCGAGTCTTT |
| | ACAGACAAGACGAGTGCAACGGTAATCTGTAGGAAGAACGCAAGCATTTCCGTCAGAG |
| | CTCAGGACCGGTACTATAGCAGTTCATGGTCTGAATGGGCTAGTGTACCTTGCAGTGG |
| | AGGTGGCGGCGGCGGCTCTCGAAATCTGCCAGTCGCTACCCCGGACCCAGGAATGTTT |
| | CCATGCCTGCACCACAGTCAGAACCTGCTCCGGGCGGTTTCCAACATGCTTCAGAAAG |
| | CGCGCCAGACCCTTGAATTTTACCCCTGCACAAGTGAAGAGATAGACCATGAAGATAT |
| | TACCAAGGATAAAACATCAACTGTAGAGGCGTGTCTCCCTCTCGAACTGACAAAGAAC |
| | GAGTCTTGTCTCAATAGTAGGGAAACTTCATTCATTACAAACGGGTCATGTCTTGCTT |
| | CAAGGAAGACCAGCTTCATGATGGCACTCTGCTTGTCTTCAATCTATGAGGATCTTAA |
| | AATGTACCAAGTAGAGTTTAAGACTATGAATGCGAAGCTCCTGATGGATCCGAAGCGG |
| | CAGATTTTTTTGGACCAGAATATGTTGGCGGTCATTGACGAACTTATGCAAGCTCTCA |
| | ATTTCAATTCAGAGACGGTTCCTCAGAAAAGCTCCTTGGAAGAGCCGGACTTCTACAA |
| | AACTAAGATCAAATTGTGTATCTTGCTCCATGCATTCCGGATACGCGCCGTGACCATT |
| | GATCGAGTAATGTCCTATTTGAATGCAAGCTAA |
| | Amino acid sequence (SEQ ID NO: 33) |
| | MCHQQLVISWFSLVFLASPLVAIWELKKDVYVVELDWYPDAPGEMVVLTCDTPEEDGI |
| | TWTLDQSSEVLGSGKTLTIQVKEFGDAGQYTCHKGGEVLSHSLLLLHKKEDGIWSTDI |
| | LKDQKEPKNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYTSSFFIRDI |
| | IKPDPPKNLQLKPLKNSRQVEVSWEYPDTWSTPHSYFSLTFCVQVQGKSKREKKDRVF |
| | TDKTSATVICRKNASISVRAQDRYYSSSWSEWASVPCSGGGGGGSRNLPVATPDPGMF |
| | PCLHHSQNLLRAVSNMLQKARQTLEFYPCTSEEIDHEDITKDKTSTVEACLPLELTKN |
| | ESCLNSRETSFITNGSCLASRKTSFMMALCLSSIYEDLKMYQVEFKTMNAKLLMDPKR |
| | QIFLDQNMLAVIDELMQALNENSETVPQKSSLEEPDFYKTKIKLCILLHAFRIRAVTI |
| | DRVMSYLNAS |
| human IL-12B/ | Coding sequence (SEQ ID NO: 34) |
| p40 (codon | ATGTGCCACCAGCAACTTGTAATAAGTTGGTTCTCACTGGTCTTCCTTGCGAGCCCGT |
| optimized) | TGGTAGCAATCTGGGAACTGAAAAAGGACGTATATGTGGTGGAGCTCGATTGGTATCC |
| | CGACGCACCCGGGGAGATGGTTGTCCTCACCTGTGATACCCCAGAGGAAGACGGTATC |
| | ACTTGGACCTTGGATCAGTCCTCCGAGGTGCTTGGAAGCGGAAAAACTCTGACGATAC |
| | AGGTGAAAGAATTCGGCGACGCGGGACAGTATACATGCCACAAAGGCGGGGAGGTACT |
| | GTCACATTCACTCCTGCTCTTGCATAAAAAGGAGGACGGGATCTGGAGCACTGATATT |
| | TTGAAAGACCAAAAAGAACCAAAAAACAAGACCTTCCTCAGGTGTGAGGCCAAGAATT |
| | ATAGTGGACGCTTCACCTGCTGGTGGCTGACCACAATTAGTACTGACTTGACTTTCTC |
| | AGTAAAGAGTTCCAGGGGGAGTAGTGACCCCCAAGGGGTAACCTGCGGGGCCGCGACT |
| | TTGTCAGCGGAACGGGTGCGAGGGGATAATAAGGAATATGAGTATTCAGTGGAGTGCC |
| | AGGAAGACTCTGCATGTCCCGCAGCAGAGGAAAGCCTGCCGATAGAGGTAATGGTTGA |
| | CGCCGTCCATAAGCTCAAATACGAGAACTACACAAGCTCTTTCTTCATACGGGACATT |
| | ATTAAGCCAGACCCCCCGAAGAATCTTCAACTTAAACCGTTGAAAAATAGTCGACAAG |
| | TGGAAGTCAGTTGGGAATATCCAGACACCTGGAGTACGCCACACAGCTATTTTTCCTT |
| | GACATTTTGCGTCCAGGTTCAGGGGAAATCAAAGCGCGAGAAGAAGGATCGAGTCTTT |
| | ACAGACAAGACGAGTGCAACGGTAATCTGTAGGAAGAACGCAAGCATTTCCGTCAGAG |
| | CTCAGGACCGGTACTATAGCAGTTCATGGTCTGAATGGGCTAGTGTACCTTGCAGT |
| | Amino acid sequence (SEQ ID NO: 35) |
| | MCHQQLVISWFSLVFLASPLVAIWELKKDVYVVELDWYPDAPGEMVVLTCDTPEEDGI |
| | TWTLDQSSEVLGSGKTLTIQVKEFGDAGQYTCHKGGEVLSHSLLLLHKKEDGIWSTDI |
| | LKDQKEPKNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYTSSFFIRDI |
| | IKPDPPKNLQLKPLKNSRQVEVSWEYPDTWSTPHSYFSLTFCVQVQGKSKREKKDRVE |
| | TDKTSATVICRKNASISVRAQDRYYSSSWSEWASVPCS |
| G₆S linker (SEQ | GGGGGGS |
| ID NO: 36) | |
| human IL-12A/ | Coding sequence (SEQ ID NO: 37) |
| p35 (codon | CGAAATCTGCCAGTCGCTACCCCGGACCCAGGAATGTTTCCATGCCTGCACCACAGTC |
| optimized) | AGAACCTGCTCCGGGCGGTTTCCAACATGCTTCAGAAAGCGCGCCAGACCCTTGAATT |
| | TTACCCCTGCACAAGTGAAGAGATAGACCATGAAGATATTACCAAGGATAAAACATCA |
| | ACTGTAGAGGCGTGTCTCCCTCTCGAACTGACAAAGAACGAGTCTTGTCTCAATAGTA |
| | GGGAAACTTCATTCATTACAAACGGGTCATGTCTTGCTTCAAGGAAGACCAGCTTCAT |
| | GATGGCACTCTGCTTGTCTTCAATCTATGAGGATCTTAAAATGTACCAAGTAGAGTTT |
| | AAGACTATGAATGCGAAGCTCCTGATGGATCCGAAGCGGCAGATTTTTTTGGACCAGA |
| | ATATGTTGGCGGTCATTGACGAACTTATGCAAGCTCTCAATTTCAATTCAGAGACGGT |
| | TCCTCAGAAAAGCTCCTTGGAAGAGCCGGACTTCTACAAAACTAAGATCAAATTGTGT |
| | ATCTTGCTCCATGCATTCCGGATACGCGCCGTGACCATTGATCGAGTAATGTCCTATT |
| | TGAATGCAAGCTAA |
| | Amino acid sequence (SEQ ID NO: 38) |
| | RNLPVATPDPGMFPCLHHSQNLLRAVSNMLQKARQTLEFYPCTSEEIDHEDITKDKTS |
| | TVEACLPLELTKNESCLNSRETSFITNGSCLASRKTSFMMALCLSSIYEDLKMYQVEF |
| | KTMNAKLLMDPKRQIFLDQNMLAVIDELMQALNFNSETVPQKSSLEEPDFYKTKIKLC |
| | ILLHAFRIRAVTIDRVMSYLNAS |
| CD3ξ domain | RVKFSRSADAPAYQQGQNQLYNELNLGRREEYDVLDKRRGRDPEMGGKPRRKNPQEGL |
| (SEQ ID NO: 39) | YNELQKDKMAEAYSEIGMKGERRRGKGHDGLYQGLSTATKDTYDALHMQALPPR |
| Truncated EGFR | RKVCNGIGIGEFKDSLSINATNIKHFKNCTSISGDLHILPVAFRGDSFTHTPPLDPQE |
| (SEQ ID NO: 40) | LDILKTVKEITGFLLIQAWPENRTDLHAFENLEIIRGRTKQHGQFSLAVVSLNITSLG |
| | LRSLKEISDGDVIISGNKNLCYANTINWKKLFGTSGQKTKIISNRGENSCKATGQVCH |
| | ALCSPEGCWGPEPRDCVSCRNVSRGRECVDKCNLLEGEPREFVENSECIQCHPECLPQ |
| | AMNITCTGRGPDNCIQCAHYIDGPHCVKTCPAGVMGENNTLVWKYADAGHVCHLCHPN |
| | CTYGCTGPGLEGCPTNGPKIPSIATGMVGALLLLLVVALGIGLFM |

The process of inserting or incorporating a nucleic acid into a cell can be via known technologies, such as transformation, transfection or transduction, without limitation. Transformation introduces recombinant plasmid DNA into competent cells that take up extracellular DNA from the environment. This process is adapted for propagation of plasmid DNA, protein production, and other applications. Transfection is the process of uptake of foreign nucleic acid by a eukaryotic cell. Transduction refers to the introduction of a recombinant viral vector particle into a target cell.

The term “vectors” refers to a nucleic acid molecule capable of transporting or mediating expression of a heterologous nucleic acid. A plasmid is a species of the genus encompassed by the term “vector.” A vector typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the cell. A vector can be a DNA or RNA vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

The present disclosure also provides polynucleotides or nucleic acid molecules encoding the chimeric receptor, optionally along with other useful components of the engineered immune cell (e.g., proinflammatory cytokine and/or kill switch).

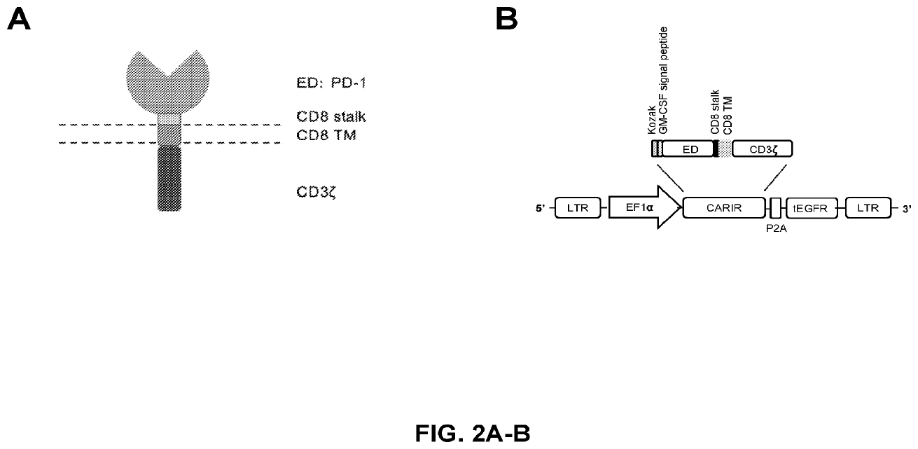

The polynucleotides of the present disclosure may encode chimeric receptor, the proinflammatory cytokine and kill switch on the same polynucleotide molecule (as exemplified in FIG. 2) or on separate polynucleotide molecules.

As illustrated in FIG. 2, the vector encodes a preprotein that includes a leader peptide from human CSF-2, a chimeric receptor that includes an extracellular domain of PD1, a CD8 hinge region, a CD8 transmembrane domain, a CD40 costimulatory domain, the CDζ activation domain, a protease digestion site P2A, a kill switch (truncated EGFR (tEGFR)), a protease digestion site T2A, and an IL-12 (including p40 and p35, connected through a short G₆S linker (SEQ ID NO:36) linker) as the additional proinflammatory cytokine. Upon protease treatments, the preprotein can be split into the chimeric receptor, the tEGFR kill switch, and the IL-12 cytokine.

The polynucleotides encoding desired proteins may be readily prepared, isolated and sequenced using conventional procedures (e.g., by using oligonucleotide probes that are capable of binding specifically to genes encoding the receptor).

Additionally, standard techniques known to those of skill in the art can be used to introduce mutations in the nucleotide sequence encoding a chimeric receptor of the present disclosure, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference chimeric receptor.

### p50 Inactivation

In some embodiments, the progenitor cell or the immune cell is engineered to be p50 deficient. NF-κB p50 (nuclear factor NF-kappa-B p105 subunit) is a Rel protein-specific transcription inhibitor, and is the DNA binding subunit of the NF-kappaB (NF-κB) protein complex. NF-κB is a transcription factor that is activated by various intra- and extra-cellular stimuli such as cytokines, oxidant-free radicals, ultraviolet irradiation, and bacterial or viral products. Activated NF-κB translocates into the nucleus and stimulates the expression of genes involved in a wide variety of biological functions. p50 is an inhibitory subunit; in the basal state p65 is held in the cytoplasm by IκB, whereas p50: p50 homo-dimers enter the nucleus, bind DNA, and repress gene expression. Absence of p50 leads to activation of proinflammatory pathways.

In some embodiments, a p50 deficient progenitor cell or immune cell is cell that has been engineered to have reduced expression or biological activity of the p50 gene. In some embodiments, the p50 gene is knocked out (p50^{-/-}). Reduced expression or biological activity or knock-out can be readily implemented with techniques well known in the art, such as CRISPR. Example CRISPR guide RNA sequences are provided in Table 3 below. In some embodiments, a single allele of the p50 gene is inactivated; in some embodiments, both alleles of the p50 gene are inactivated.

TABLE 3
Example Guide Sequences for CRISPR

| SEQ ID NO: | Guide RNA Sequence (without PAM) |
|---|---|
| 44 | ACTGGAAGCACGAATGACAG (human) |
| 45 | ATGGGCCTTCACACACATAG (mouse) |
| 46 | TACCCGACCACCATGTCCTT (human) |
| 47 | ATATAGATCTGCAACTATGT (human) |

### Preparation and Expansion of Progenitor Cells

The immune cell of the disclosure can be obtained from a commercial source, a donor subject, or a patient who desires a treatment (autologous). Alternatively, the immune cell can be generated in vitro or ex vivo from a progenitor cell. Example progenitors include hematopoietic stem cells from the bone marrow, mobilized hematopoietic stem cells from the peripheral blood, and induced pluripotent stem cells (iPSCs).

The progenitor cells are preferably CD34⁺ cells. Cell surface markers such as CD34, in some embodiments, are used to isolate or enrich such progenitor cells from the source material. Methods of preparing iPSCs are also known in the art. Induced pluripotent stem cells (iPSCs) are a type of pluripotent stem cell that can be generated directly from a somatic cell, such as a skin cell, a peripheral blood cell, or a renal epithelial cell. Human CD34⁺ cells can also be obtained from bone marrow or peripheral blood.

The total number of cells can be increased to prepare one or more effective doses of the engineered cells for therapies. Such an expansion step can be carried out at any time in the process, but for multiple rounds. In the illustration of FIG. 1, the expansion of progenitor cells is initiated prior to gene editing and transduction. In alternatively embodiments, the expansion can be carried out after gene editing and transduction.

Expansion of the cells can be carried out under hypoxic conditions. It is contemplated that hypoxic conditions promote proliferation of the progenitor or immune cells.

Methods of providing hypoxia for cell culturing are known in the art. In one example, a cobalt salt, such as cobalt chloride hexahydrate (CoCl₂·6H₂O), is added to the culture media to induce hypoxia. An example procedure is as follows. A 25 mM CoCl₂ stock solution can be prepared in sterile water. The stock solution is added to the cell culture media to arrive at a final concentration of 50˜150 μM. The culture is incubated in a conventional incubator (e.g., 37° C.; 5% CO₂). In some embodiments, the cobalt concentration is 10 to 500 μM. In some embodiments, the cobalt concentration is 20 to 400 μM, 40 to 300 μM, 50 to 200 μM, 50 to 150 μM, 80 to 120 μM, 90 to 110 μM, or at about 100 μM.

In another embodiment, hypoxia is induced in a modular chamber that provides reduced supply of oxygen. The modular chamber may be an incubator, a flask, without limitation. In some embodiment, the chamber is supplied with air that has reduced oxygen concentration. The normal oxygen concentration is about 20%. A reduced oxygen concentration, in some embodiments, is not higher than 15%, 12%, 10%, 9%, 8%, 7%, 6%, 5%, 4%, 3%, 2%, 1%, 0.5%, 0.2% or 0.1%. In some embodiments, the chamber includes about 80%, 85%, 90%, or 95% nitrogen. In some embodiments, the chamber includes about 5%, 2% or 1% CO₂.

In some embodiments, the hypoxic culturing is carried out for at least 2 hours, or at least 4 hours, 6 hours, 12 hours, 18 hours, 24 hours, 36 hours, 48 hours, or 72 hours. In some embodiments, the cells are kept in a hypoxic condition after proliferation.

In some embodiments, the culture media for cell expansion includes one or more ingredient useful for cell growth or proliferation. Examples include stem cell factor (SCF), thrombopoietin (TPO) and FMS-like tyrosine kinase 3 ligand (FLT3L).

### Cell Differentiation

The progenitors, whether prior to or following p50 inactivation/receptor transduction, but preferably after cell expansion, can be differentiated into immune cells. Example immune cells include myeloid cells, natural killer (NK) cells, T cells, tumor infiltrating lymphocytes, and natural killer T (NKT) cells. In some embodiments, the immune cell is a myeloid cell, in particular an immature myeloid cell (IMC).

Myeloid cells are produced by hematopoietic stem cells. Myeloid cells are also progenitor cells which can produce different types of blood cells including monocytes, macrophages, neutrophils, basophils, eosinophils, erythrocytes, dendritic cells, megakaryocytes, and platelets. Myeloid cells originate in bone marrows. Myeloid cells encompass circulating progenitor monocytes and tissue resident macrophage cells, including hepatic Kupffer cells, lymph-associated macrophages in spleen and lymph nodes, Langerhans cells in the skin, pulmonary alveolar macrophages, and highly specialized dendritic cells found primarily along mucosal surfaces.

“Immature myeloid cells” (IMC), “early myeloid cells,” “myeloid suppressive cells,” or “myeloid-derived suppressor cells” (MDSCs), are progenitor cells present in the bone marrow and spleen of healthy subjects which can differentiate into mature myeloid cells under normal conditions. These cells are associated with immune suppression during viral infection, transplantation, UV irradiation and cyclophosphamide (CTX) treatment. It has also been shown that the accumulation of IMC within the tumor microenvironment correlates with a poor prognosis. The instant inventors have the insight that immunotherapy with such cells can promote penetration into the tumor microenvironment.

Differentiation, or more specifically limited differentiation, of the progenitor cells can be carried out in suitable culture media. In some embodiments, the culture media include macrophage colony-stimulating factor (M-CSF). In some embodiments, the differentiation in the presence of M-CSF is for at least 1 hour, 2 hours, 4 hours, 6 hours, 12 hours, 24 hours, or for up to 12 hours, 24 hours, 36 hours, 48 hours, or 72 hours.

In some embodiments, the immune cell is further engineered to produce a proinflammatory cytokine. Example proinflammatory cytokines include the IL-1 family (e.g., IL18, IL18BP, IL1A, IL1B, IL1F10, IL1F3/IL1RA, IL1F5, IL1F6, IL1F7, IL1F8, IL1RL2, IL1F9, and IL33), IL-1 receptors (e.g., IL18R1, IL18RAP, IL1R1, IL1R2, IL1R3, IL1R8, IL1R9, IL1RL1, and SIGIRR), the TNF family (BAFF, 4-1BBL, TNFSF8, CD40LG, CD70, CD95L/CD178, EDA-A1, TNFSF14, LTA/TNFB, LTB, TNFa, TNFSF10, TNFSF11, TNFSF12, TNFSF13, TNFSF15, and TNFSF4), Interferons (IFN) (e.g., IFNA1, IFNA10, IFNA13, IFNA14, IFNA2, IFNA4, IFNA7, IFNB1, IFNE, IFNG, IFNZ, IFNA8, IFNA5/IFNaG, and IFNω/IFNW1), the IL6 family (e.g., CLCF1, CNTF, IL11, IL31, IL6, Leptin, LIF, OSM, IL6 Receptor, CNTFR, IL11RA, IL6R, LEPR, LIFR, OSMR, and IL31RA), chemokines (e.g., CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, XCL1, XCL2, FAM19A1, FAM19A2, FAM19A3, FAM19A4, and FAM19A5).

In some embodiments, the proinflammatory cytokine is IL-12, IFN-γ, TNF-α, and/or IL-1β. In some embodiments, the expression of the cytokine is constitutive. In some embodiments, the expression of the cytokine is inducible.

It is contemplated that expression of the proinflammatory cytokine, such as IL-12, enable the engineered immune cells to reprogram and overcome the immunosuppressive TME. Further, the additional expression of the proinflammatory cytokine can enhance tumor antigen presentation, increase tumor-specific cytotoxic T cells activation, and prevent or reduce tumor metastasis.

In some embodiments, the immune cell further expresses a kill switch (or safety module). The kill switch allows the engineered immune cell to be killed or turned off when needed. In some embodiments, the kill switch is a human HSV-TK, a truncated EGFR (tEGFR, e.g., SEQ ID NO:40), or a CD20 protein or fragment. In the case of unacceptable toxicity, the immune cells can be eliminated through administration of a corresponding drug (e.g., ganciclovir) or depleting antibody, (e.g., Cetuximab or Rituximab).

Collection of CD34⁺ cells from adult bone marrow can be challenging and the amount collected is typically low. An alternative approach is to use mobilized peripheral blood (MPB). MPB is collected from healthy donors that are injected with Granulocyte-Colony Stimulating Factor (G-CSF), Plerixafor, or a combination of Plerixafor and G-CSF. These mobilization agents increase circulating leukocytes and stimulate the bone marrow to produce a large number of hematopoietic stem cells, which are mobilized into the bloodstream, allowing for large quantities of stem cells and MNCs to be collected from a single donor.

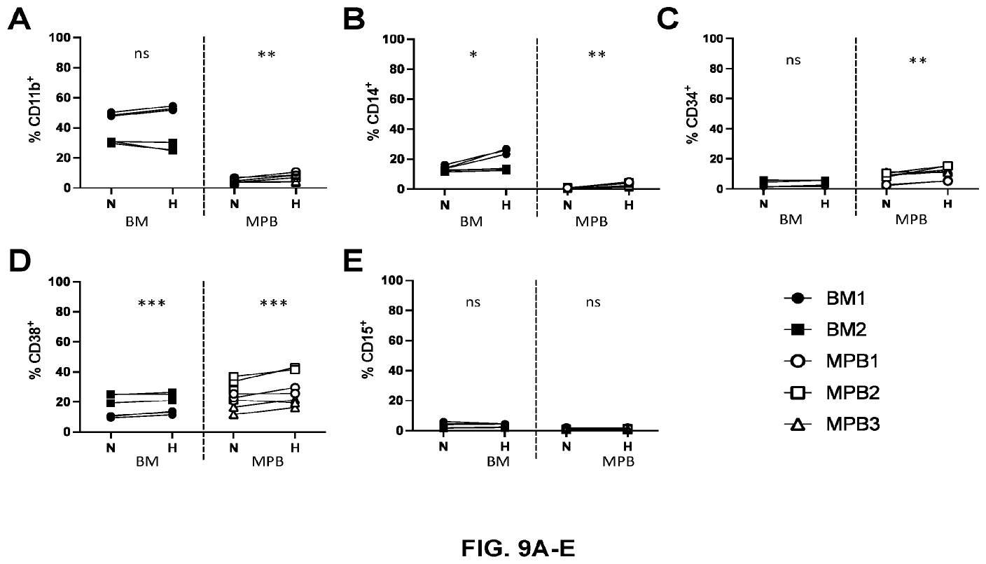

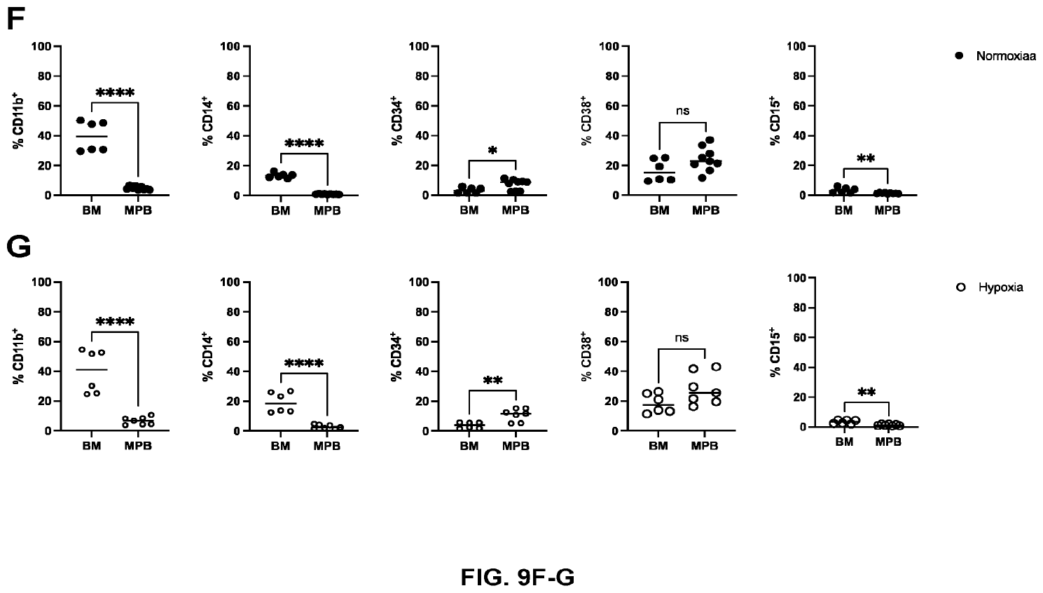

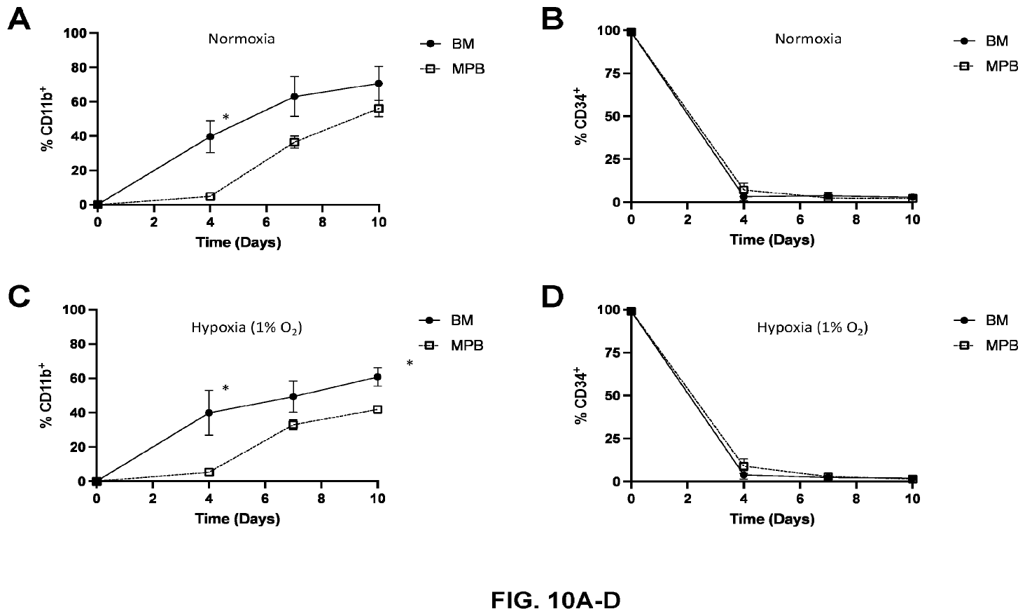

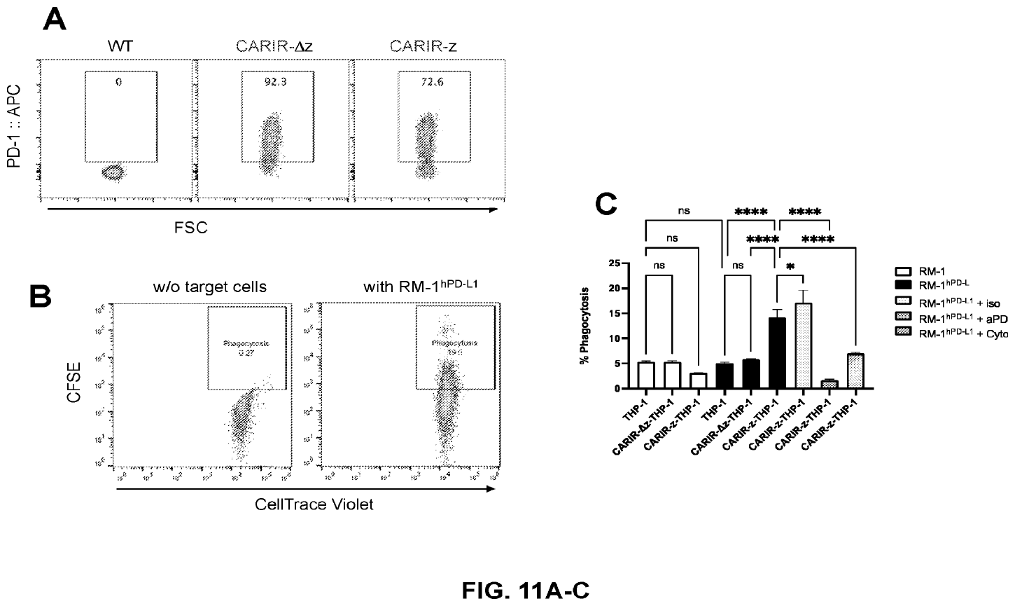

In a surprising discovery, however, the instant inventors observed that more myeloid cells can be generated from differentiation of bone marrow-derived human CD34⁺ cells than from MPB-derived human CD34⁺ cells, under both normoxia and hypoxia conditions (FIGS. 9 and 10). In some embodiments, therefore, the bone marrow is the preferred source of progenitor cells.

### Cancer Treatment

As described herein, the engineered immune cells of the present disclosure can be used in certain treatment methods. Accordingly, one embodiment of the present disclosure is directed to immune cell-based therapies which involve administering the immune cells of the disclosure to a patient such as an animal, a mammal, and a human for treating one or more of the disorders or conditions described herein.

In some embodiments, the cancer being treated expresses a ligand corresponding to the extracellular targeting domain of the chimeric receptor. For instance, if the cancer cells express PD-L1, then a suitable chimeric receptor includes an extracellular targeting domain of PD1. Likewise, if the cancer cells express CD47, then a suitable chimeric receptor includes an extracellular targeting domain of SIRPα.

Cancers that can be suitable treated by the present technology include bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, esophageal cancer, ovarian cancer, renal cancer, and small cell lung cancer. Accordingly, the presently disclosed antibodies can be used for treating any one or more such cancers, in particular non-small cell lung cancer (NSCLC), small cell lung cancer, prostate cancer, pancreatic ductal carcinoma, neuroblastoma, glioblastoma, ovarian cancer, melanoma, and breast cancer. In some embodiments, the cancer is metastatic cancer.

Additional diseases or conditions associated with increased cell survival, that may be treated or prevented include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, the immune cell is isolated from the cancer patient him- or her-self. In some embodiments, the immune cell is provided by a donor or from a cell bank. When the cell is isolated from the cancer patient, undesired immune reactions can be minimized. The isolated immune cell can then be transduced with the polynucleotides or vectors of the present disclosure to prepare the engineered immune cell.

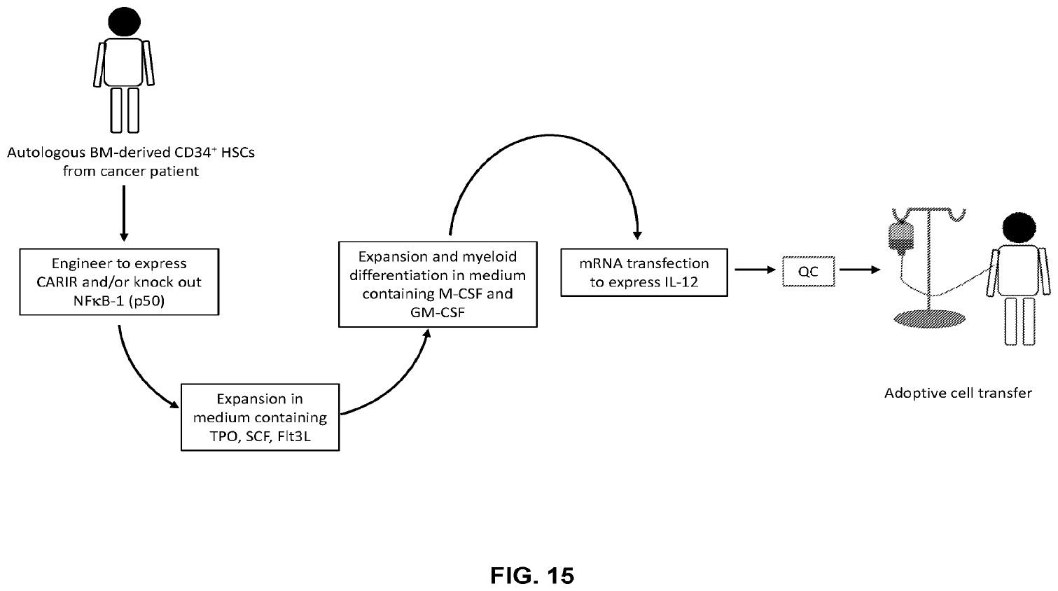

An example adoptive engineered-myeloid cell therapy for treating cancer is illustrated in FIG. 15. At a first step, autologous HSCs are acquired from a cancer patient. Within the HSCs, the p50 gene is inactivated and a CARIR construct is introduced. Then, the transduced HSCs are optionally expanded and differentiated into immature myeloid cells (IMC) or other types of myeloid cells or immune cells, which are transferred back to the patient for treatment.

### Combination Therapies

In a further embodiment, the compositions of the disclosure are administered in combination with a different antineoplastic agent. Any of these agents known in the art may be administered in the compositions of the current disclosure.

In one embodiment, compositions of the disclosure are administered in combination with a chemotherapeutic agent. Chemotherapeutic agents that may be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, fludarabine, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In one embodiment, compositions of the disclosure are administered in combination with a checkpoint inhibitor, such as anti-PD-1/PD-L1 or anti-CTLA4 antibodies. For example, if the engineered immune cell targets PD-L1, the checkpoint inhibitor may target CTLA-4. Likewise, if the engineered immune cell targets CTLA-4, the checkpoint inhibitor may target PD-1 or PD-L1.

In one embodiment, compositions of the disclosure are administered in combination with another cell therapy agent, such as TILs, CAR-T, CAR-NK, CAR-γδT, T-cell antigen coupler (TAC)-T.

In additional embodiments, the compositions of the disclosure are administered in combination with other therapeutic or prophylactic regimens, such as, for example, radiation therapy.

Combination therapies are also provided, which includes the use of one or more of the immune cells of the present disclosure along with a second anticancer (chemotherapeutic) agent. Chemotherapeutic agents may be categorized by their mechanism of action into, for example, anti-metabolites/anti-cancer; purine analogs, folate antagonists, and related inhibitors, antiproliferative/antimitotic agents, DNA damaging agents, antibiotics, enzymes such as L-asparaginase which systemically metabolizes L-asparagine and deprives cells which do not have the capacity to synthesize their own asparagine, antiplatelet agents, antiproliferative/antimitotic alkylating agents, antiproliferative/antimitotic antimetabolites, platinum coordination complexes, hormones, hormone analogs, anticoagulants, fibrinolytic agents, antimigratory agents, antisecretory agents, immunosuppressives, angiotensin receptor blockers, nitric oxide donors, cell cycle inhibitors and differentiation inducers, topoisomerase inhibitors, growth factor signal transduction kinase inhibitors, without limitation.

Additional examples include alkylating agents, alkyl sulfonates, aziridines, emylerumines and memylamelamines, acetogenins, nitrogen mustards, nitrosoureas, anti-metabolites, folic acid analogs, purine analogs, pyrimidine analogs, androgens, anti-adrenals, folic acid replinishers, trichothecenes, and taxoids, platinum analogs.

In one embodiment, the compounds and compositions described herein may be used or combined with one or more additional therapeutic agents. The one or more therapeutic agents include, but are not limited to, an inhibitor of Abl, activated CDC kinase (ACK), adenosine A2B receptor (A2B), apoptosis signal-regulating kinase (ASK), Auroa kinase, Bruton's tyrosine kinase (BTK), BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, FYN, glycogen synthase kinase (GSK), HCK, histone deacetylase (HDAC), IKK such as IKKβε, isocitrate dehydrogenase (IDH) such as IDH1, Janus kinase (JAK), KDR, lymphocyte-specific protein tyrosine kinase (LCK), lysyl oxidase protein, lysyl oxidase-like protein (LOXL), LYN, matrix metalloprotease (MMP), MEK, mitogen-activated protein kinase (MAPK), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase STK, signal transduction and transcription (STAT), SRC, serine/threonine-protein kinase (TBK) such as TBK1, TIE, tyrosine kinase (TK), vascular endothelial growth factor receptor (VEGFR), YES, or any combination thereof.

For any of the above combination treatments, the engineered immune cell can be administered concurrently or separately from the other anticancer agent. When administered separately, the engineered immune cell can be administered before or after the other anticancer agent.

### EXPERIMENTAL EXAMPLES

### Example 1. Transduction and Expression of PD-1 CARIR in Human Cells

This example tested lentiviral mediated transduction to and expression in human monocytic THP-1 cells of a Chimeric CAR-like Immune Receptor (CARIR).

A lentiviral vector was constructed to encode the CARIR protein, whose structure is illustrated in FIG. 2A. The CARIR included an extracellular domain (ED), a CD8 hinge region, a CD8 transmembrane domain, and the CD3 activation domain. The ED was derived from PD-1. In addition, the lentiviral vector included a protease digestion site P2A, and a kill switch (truncated EGFR (tEGFR)). Upon protease treatments, the expressed preprotein can be split into the chimeric receptor and the tEGFR kill switch.

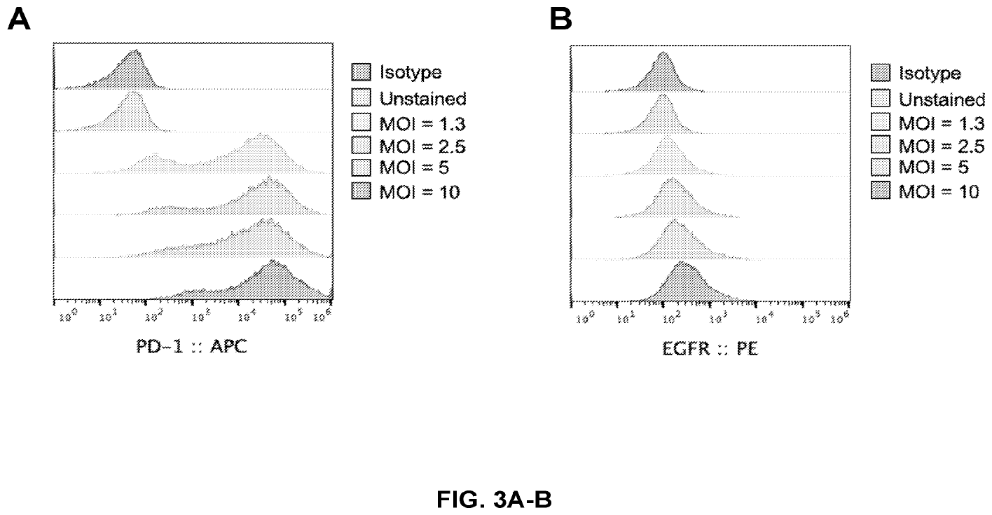

THP-1 cells were transduced with lentiviral vector encoding PD-1 CARIR at certain multiplicity of infection (MOI), including 1.3, 2.5, 5, and 10. The cells were gated on live and singlets. The expression of the CARIR was measured by flow cytometry through detecting surface expression of PD-1. Offset histograms confirmed CARIR expression (FIG. 3A) and the expression of truncated EGFR (FIG. 3B) which serve as a safety kill switch.

### Example 2. Transduction and Expression of Additional CARIR in Human Cells

This example shows that CARIR with other types of extracellular domains can likewise prepared and expressed in human cells.

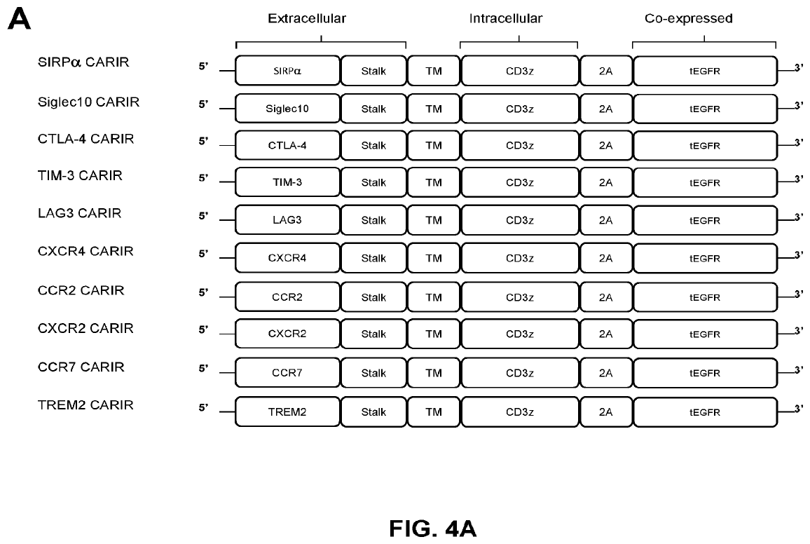

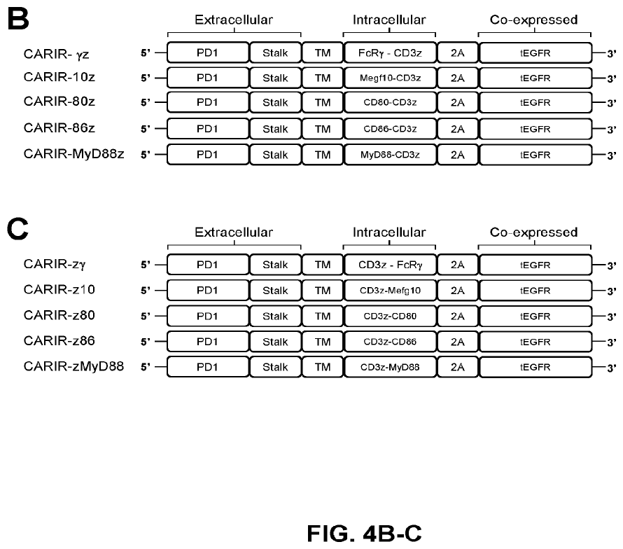

Besides PD-1, the extracellular fragments of many other receptors can also be used to construct CARIR. Some of the additional example CARIR molecules are illustrated in FIG. 4A, with extracellular domains derived from receptors that are relevant in cancer immunotherapy, such as SIRPα, Siglec10, CTLA-4, TIM-3, LAG3, CXCR4, CCR2, CXCR2, CCR7, and TREM2. Also, besides just the CD35 activation domain, the CARIR can include other intracellular domains, such as those illustrated in FIG. 4B-C.

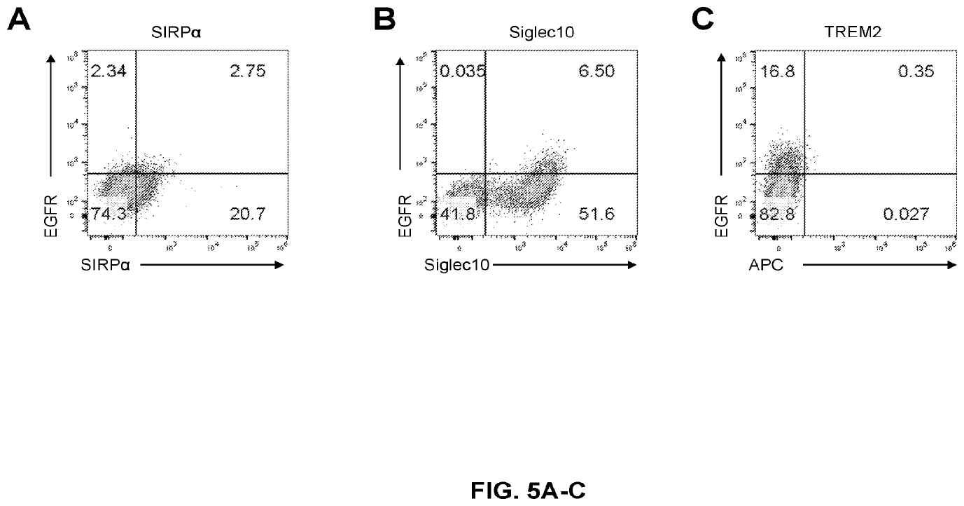

Some of these additional example CARIR molecules were tested in this example. THP-1 cells were transduced with lentiviral vector encoding CARIR that included an extracellular domain of SIRPα, Siglec10 or TREM2. The cells were gated on live and singlets. The expression of the CARIR was measured by flow cytometry through detecting surface expression of the corresponding extracellular domain. Dot plots show CARIR and EGFR surface expression in CARIR transduced THP-1 cells (FIG. 5A-C).

### Example 3. p50-knockout HSCs

This example prepared hematopoietic stem cells (HSCs) in which the NFκB-1 (p50) gene was knocked out through the CRISPR/Cas9 approach.

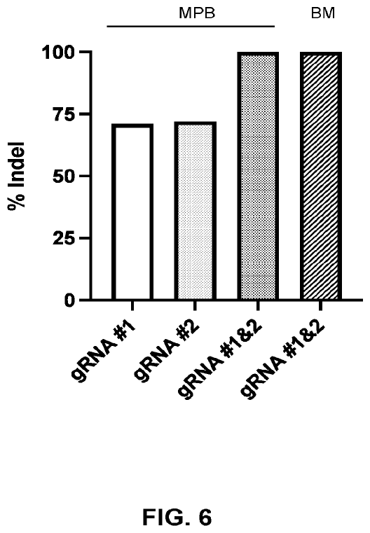

Human mobilized peripheral blood (MPB) or bone marrow (BM)-derived CD34⁺ hematopoietic stem cells (HSCs) were electroporated with ribonucleoprotein (RNP) complex containing recombinant Cas9 protein as well as guide RNA #1 (gRNA #1, target: TACCCGACCACCATGTCCTT, SEQ ID NO:46) and/or guide RNA #2 (gRNA #2, target: ATATAGATCTGCAACTATGT, SEQ ID NO:47). The % indel among the NFκB-1 (p50) gene was measured by TIDE analysis 6 days later, and the results are shown in FIG. 6.

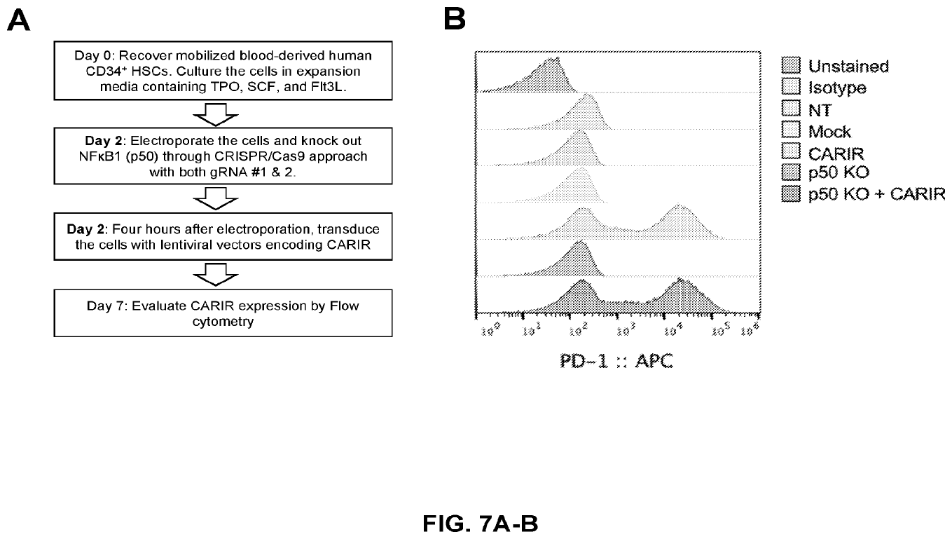

The p50-knockout HSCs were then transduced with, on the same day, constructs encoding the PD-1 CARIR. The procedure is illustrated in FIG. 7A. Offset histograms show CARIR (PD-1) expression on HSCs with or without NF-kB1 (p50) knock out (FIG. 7B).

### Example 4. Expansion and Differentiation of Transduced Cells

This example measured expansion and differentiation of cells transduced with CARIR under different conditions.

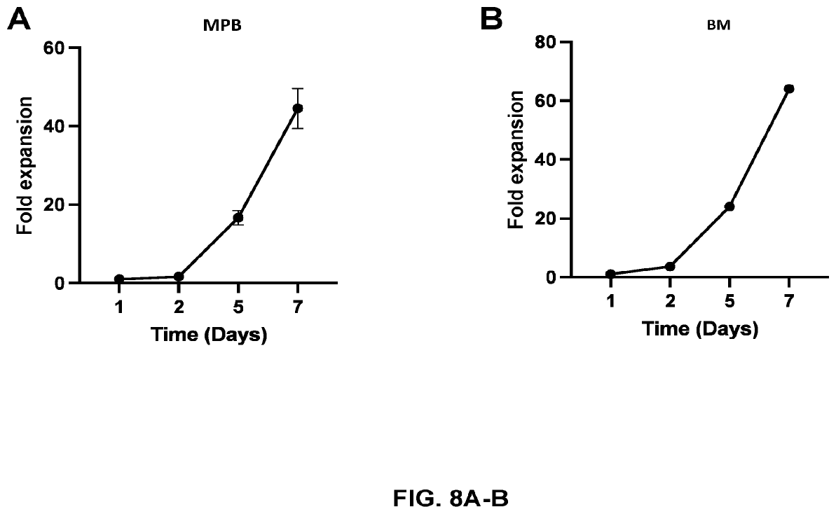

The transduced human CD34⁺ hematopoietic stem cells were expanded in vitro for a week in culture medium containing TPO, SCF, Flt3L, and UM171. The number of live cells were counted at the indicated time points. Average numbers of the cells at a few time points are shown in FIG. 8A-B.

The BM and MPB-derived human CD34⁺ HSCs were differentiated at either normoxia or hypoxia conditions. The cells were cultured in myeloid differentiation medium for 4 days in incubators that maintain either normoxia (N, 20% O₂) or hypoxia (H, 1% O₂) conditions. The cells were then analyzed by flow cytometry for relevant markers, including CD11b, CD14, CD34, CD38, and C15.

Pairwise comparison of differentiation between normoxia (N) versus hypoxia (H) culture conditions was made for these markers, and are shown in FIG. 9A-E (CD11b (A), CD14 (B), CD34 (C), CD38 (D), or CD15 (E). Summarized data in FIG. 9F show the frequency of cells among BM versus MPB-derived CD34⁺ HSCs following myeloid differentiation under normoxia condition. Summarized data in FIG. 9G show the frequency of cells among BM versus MPB-derived CD34⁺ HSCs following myeloid differentiation under hypoxia condition. These comparisons show that more myeloid cells differentiated from BM—than from MPB-derived human CD34⁺ HSCs under either normoxia or hypoxia conditions.

In another experiment, MPB- or (BM)-derived human CD34⁺ HSCs were plated in ultralow attachment plates in myeloid differentiation medium containing M-CSF and GM-CSF. The cells were cultured in the incubator that either maintain normoxia (20% O₂) or hypoxia (1% O₂) as indicated. On day 4, 7, and 10, the cells were analyzed by flow cytometry for cell surface markers CD11b and CD34. FIG. 10A-D shows the percentage of CD11b⁺ (A and C), and CD34⁺ (B and D) myeloid cells differentiated under normoxia (A and B) or hypoxia conditions (C and D) over the time course. Again, more myeloid cells resulted from differentiation of BM-versus MPB-derived human CD34⁺ HSCs over a time course of 10 days.

### Example 5. Phagocytosis by CARIR-Expressing Monocytic Cells

This example measured phagocytosis of CARIR-expressing monocytic cells.

Phagocytosis assay was utilized to evaluate the functionality of CARIR. The CellTrace Violet labeled effector THP-1, CARIR-Δz THP-1 (lacking CD3ζ signaling domain in the CARIR), or CARIR-z THP-1 cells were pretreated with 1 ng/ml PMA for 24 hours. The effector cells were then co-cultured for 4 hours with CFSE labeled RM-1 or RM-1^{hPD-L1} (RM-1 cells that engineered to overexpress human PD-L1) target cells at the effector to target ratio of 5:1, in the presence or absence of 2 μM cytochalasin D (cyto), 10 μg/ml pembrolizumab biosimilar anti-PD-1 antibody (aPD1), or human IgG4 isotype control (iso). The % phagocytosis was assessed by flow cytometry. FIG. 11A shows CARIR transduction efficiency in THP-1 cells as evaluated by flow staining for PD-1. The cells were gated on live, singlets. FIG. 11B shows example flow dot plots showing the % phagocytosis by CARIR-z-THP-1 cells in the presence or absence of RM-1^{hPD-L1} target cells. FIG. 11C presents summary comparison charts. CARIR expression in human monocytic THP-1 cells significantly increased phagocytosis on RM-1^{hPD-L1} target cells.

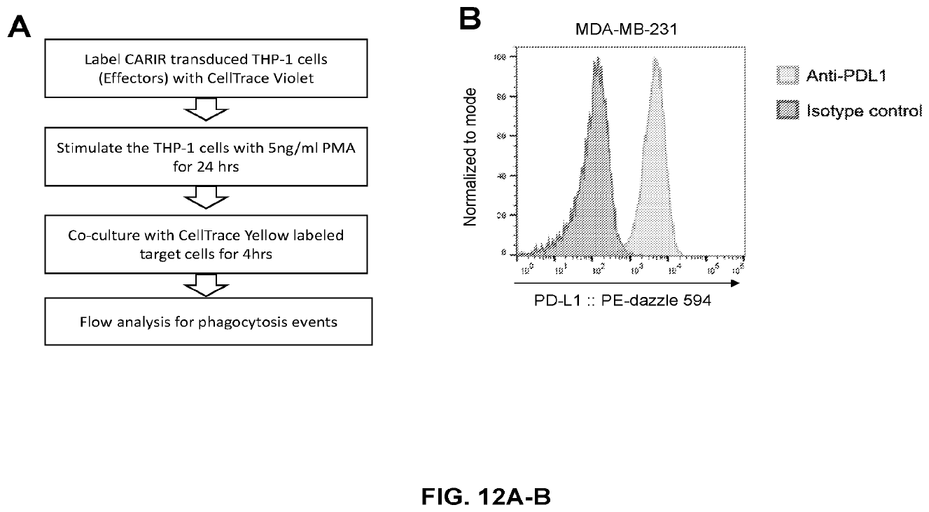

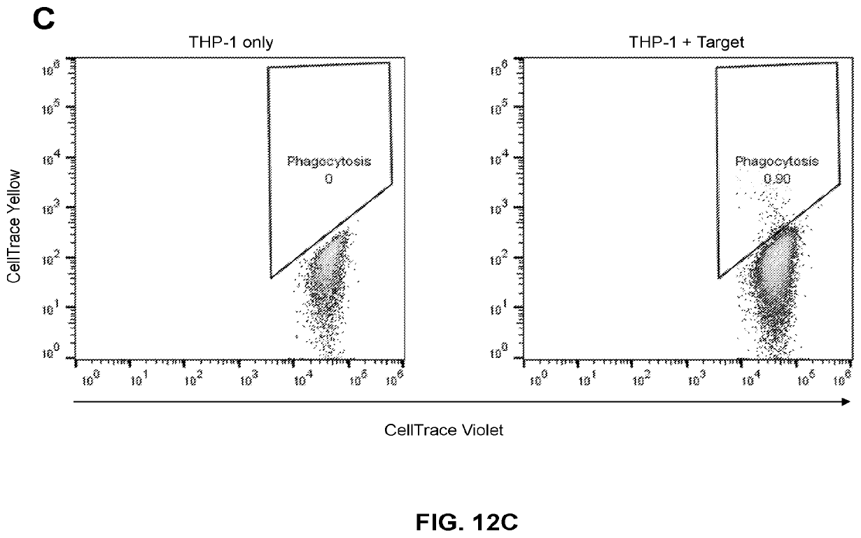

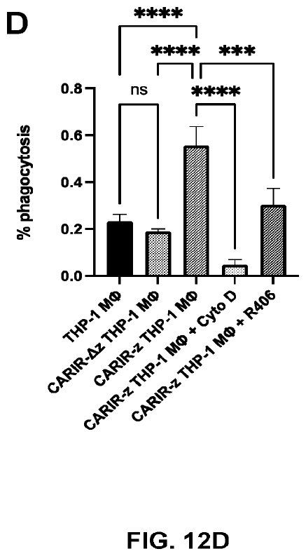

The phagocytosis assay was also performed with human triple negative breast cancer cells, and as shown in FIG. 12A-D, CARIR expression in human monocytic THP-1 cells significantly increased their phagocytosis on PD-L1⁺. To serve as the effectors, human monocytic THP-1 cells were engineered to express either CARIR-Δz (a PD-1 CARIR that lacks CD3ζ signaling domain) or CARIR-z through lentiviral transduction, and then differentiated to macrophages by PMA treatment. MDA-MB-231 tumor cells that express PD-L1 were used to serve as the target cells.

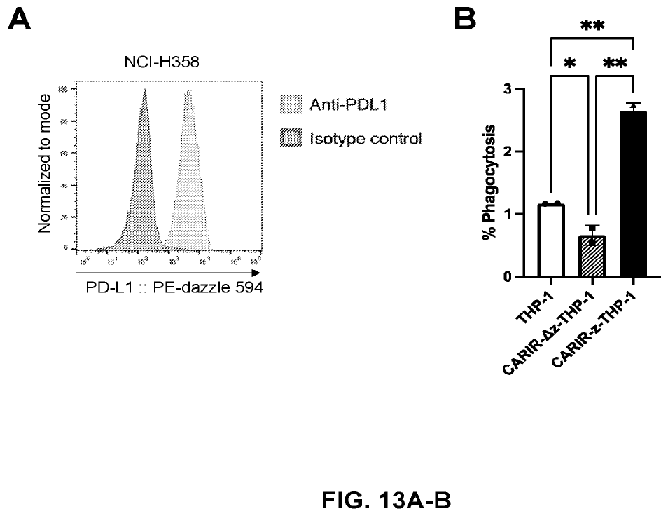

Likewise, as shown in FIG. 13A-B, CARIR expression significantly increased the phagocytosis of human THP-1 macrophages on PD-L1⁺ NCI-H358 human lung cancer cells.

### Example 6. In Vivo Testing CARIR-Expressing Myeloid Cells

This example conducted in vivo testing for myeloid cells engineered to express CARIR molecules

A schematic timeline for the in vivo experiment is provided in FIG. 14A. Syngeneic Balb/c mice were subcutaneously implanted with 5×10⁴ 4T1 breast cancer cells on day −7. Starting on day 0, the mice were treated with 3 weekly dose of 10×10⁶ engineered mouse immature myeloid cells expressing murine analog of CARIR (CARIR-IMC) or with NFκB-1 (p50) knocked out (p50^{-/-}-IMC) or PBS as indicated.

The results are shown in FIG. 14B-D, with tumor volume measurements (B) probability of survival (C) and body weight over the course of the experiment (D). Infusion of engineered myeloid cells significantly slowed 4T1 tumor growth and prolonged survival in syngeneic mouse model.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for preparing an immune cell that expresses a chimeric receptor, comprising introducing to a progenitor cell or an immune cell a polynucleotide that encodes a chimeric receptor comprising, from the N-terminus to the C-terminus, an extracellular domain of a receptor to a tumor-associated ligand, a transmembrane domain, a costimulatory domain, and a CD3ζ intracellular domain.

2. The method of claim 1, further comprising culturing the progenitor cell or immune cell in a medium under a hypoxic condition.

3. The method of claim 2, wherein the hypoxic condition is induced by a cobalt salt in the medium.

4. The method of claim 3, wherein the medium comprises 20 μM to 200 μM CoCl₂, preferably 50 μM to 150 μM CoCl₂.

5. The method of claim 2, wherein the hypoxic condition is induced by placing the medium in a chamber having no more than 10% oxygen in the air, preferably no more than 5% oxygen in the air.

6. The method of claim 2, wherein the culturing under the hypoxic condition is for at least 24 hours, preferably at least 48 hours.

7. The method of claim 1, wherein the progenitor cell or immune cell is p50 deficient.

8. The method of claim 7, wherein the progenitor cell or immune cell does not express an active p50 or has reduced p50 activity.

9. The method of claim 1, wherein the progenitor cell is selected from the group consisting of a CD34⁺ hematopoietic stem cell from bone marrow, a mobilized CD34⁺ hematopoietic stem cell from peripheral blood, and an induced pluripotent stem cell (iPSC).

10-14. (canceled)

15. The method of claim 1, wherein the extracellular domain comprises a ligand-binding domain.

16. The method of claim 1, wherein the receptor is selected from the group consisting of PD1, SIRPα, Siglec-10, CTLA-4, CXCR-4, CCR-2, CXCR2, CCR7, CD80, TIM-3, LAG3 and TREM2.

17. The method of claim 16, wherein the extracellular domain of the receptor comprises an amino acid sequence selected from the group consisting of SEQ ID NO:2, 5, 8, 11, 18, 20, 22, 24, and 42.

18. The method of claim 1, wherein the costimulatory domain is a signaling domain of a protein selected from the group consisting of CD28, CD27, OX40, CD40, CD80, CD86, and 4-1BB.

19. The method of claim 1, wherein the progenitor cell or immune cell further is transduced with a polynucleotide encoding a proinflammatory cytokine.

20. The method of claim 19, wherein the proinflammatory cytokine is selected from the group consisting of IL-12, IFN-γ, TNF-α, and IL-1β.

21. The method of claim 1, wherein the progenitor cell or immune cell further comprises a kill switch.

22. The method of claim 21, wherein the kill switch is selected from the group consisting of HSV-TK, truncated EGFR (tEGFR), and CD20.

23. The method of claim 1, further comprising administering the immune cell to a patient having cancer.

24-25. (canceled)

26. A method for preparing an immune cell, comprising:
decreasing the biological activity of the p50 gene in a progenitor cell;
expanding the progenitor cell under a hypoxic condition;
transducing to the progenitor cell a polynucleotide encoding a chimeric receptor; and
differentiating the progenitor cell into an immature myeloid cell, wherein the chimeric receptor comprises, from the N-terminus to the C-terminus, an extracellular domain of a receptor to a tumor-associated ligand, a transmembrane domain, a costimulatory domain, and a CD3ζ intracellular domain.

27. The method of claim 26, further comprising administering the immature myeloid cell to a patient having cancer.

28-29. (canceled)


SEQUENCE LISTING (47 sequences)

SEQ ID NO: 1 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 30 aa, synthetic construct
MSPSNQTDKLAAFPEDRSQPGQDCRFRVTQ

SEQ ID NO: 3 - protein, 101 aa, synthetic construct
MSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 4 - protein, 504 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 5 - protein, 103 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVE

SEQ ID NO: 6 - protein, 235 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRA

SEQ ID NO: 7 - protein, 697 aa, synthetic construct
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 8 - protein, 129 aa, synthetic construct
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQK

SEQ ID NO: 9 - protein, 238 aa, synthetic construct
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALE

SEQ ID NO: 10 - protein, 223 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 11 - protein, 99 aa, synthetic construct
RGIASFVCEYASPGKATEVRVTVLRQADSQVTEVCAATYMMGNELTFLDDSICTGTSSGNQVNLTIQGLRAMDTGLYICKVELMYPPPYYLGIGNGTQI

SEQ ID NO: 12 - protein, 301 aa, synthetic construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 13 - protein, 180 aa, synthetic construct
EVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 14 - protein, 525 aa, synthetic construct
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 15 - protein, 237 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTV

SEQ ID NO: 16 - protein, 353 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVS

SEQ ID NO: 17 - protein, 352 aa, synthetic construct
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 18 - protein, 38 aa, synthetic construct
MEGISIYTSDNYTEEMGSGDYDSMKEPCFREENANFNK

SEQ ID NO: 19 - protein, 360 aa, synthetic construct
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 20 - protein, 37 aa, synthetic construct
LSTSRSRFIRNTNESGEEVTTFFDYDYGAPCHKFDVK

SEQ ID NO: 21 - protein, 360 aa, synthetic construct
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 22 - protein, 47 aa, synthetic construct
EDFNMESDSFEDFWKGEDLSNYSYSSTLPPFLLDAAPCEPESLEINK

SEQ ID NO: 23 - protein, 378 aa, synthetic construct
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 24 - protein, 34 aa, synthetic construct
QDEVTDDYIGDNTTVDYTLFESLCSKKDVRNFKA

SEQ ID NO: 25 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 26 - protein, 17 aa, synthetic construct
MWLQSLLLLGTVACSIS

SEQ ID NO: 27 - protein, 49 aa, synthetic construct
KPTTTPAPRPPTPAPTIASQPLSLRPEACRPAAGGAVHTRGLDFACDIY

SEQ ID NO: 28 - protein, 21 aa, synthetic construct
IWAPLAGTCGVLLLSLVITLY

SEQ ID NO: 29 - protein, 19 aa, synthetic construct
ATNFSLLKQAGDVEENPGP

SEQ ID NO: 30 - protein, 18 aa, synthetic construct
EGRGSLLTCGDVEENPGP

SEQ ID NO: 31 - protein, 62 aa, synthetic construct
KKVAKKPTNKAPHPKQEPQEINFPDDLPGSNTAAPVQETLHGCQPVTQEDGKESRISVQERQ

SEQ ID NO: 32 - DNA, 1599 bp, synthetic construct
  source 1..1599: mol_type = other DNA
atgtgccaccagcaacttgtaataagttggttctcactggtcttccttgcgagcccgttggtagcaatctgggaactgaaaaaggacgtatatgtggtggagctcgattggtatcccgacgcacccggggagatggttgtcctcacctgtgataccccagaggaagacggtatcacttggaccttggatcagtcctccgaggtgcttggaagcggaaaaactctgacgatacaggtgaaagaattcggcgacgcgggacagtatacatgccacaaaggcggggaggtactgtcacattcactcctgctcttgcataaaaaggaggacgggatctggagcactgatattttgaaagaccaaaaagaaccaaaaaacaagaccttcctcaggtgtgaggccaagaattatagtggacgcttcacctgctggtggctgaccacaattagtactgacttgactttctcagtaaagagttccagggggagtagtgacccccaaggggtaacctgcggggccgcgactttgtcagcggaacgggtgcgaggggataataaggaatatgagtattcagtggagtgccaggaagactctgcatgtcccgcagcagaggaaagcctgccgatagaggtaatggttgacgccgtccataagctcaaatacgagaactacacaagctctttcttcatacgggacattattaagccagaccccccgaagaatcttcaacttaaaccgttgaaaaatagtcgacaagtggaagtcagttgggaatatccagacacctggagtacgccacacagctatttttccttgacattttgcgtccaggttcaggggaaatcaaagcgcgagaagaaggatcgagtctttacagacaagacgagtgcaacggtaatctgtaggaagaacgcaagcatttccgtcagagctcaggaccggtactatagcagttcatggtctgaatgggctagtgtaccttgcagtggaggtggcggcggcggctctcgaaatctgccagtcgctaccccggacccaggaatgtttccatgcctgcaccacagtcagaacctgctccgggcggtttccaacatgcttcagaaagcgcgccagacccttgaattttacccctgcacaagtgaagagatagaccatgaagatattaccaaggataaaacatcaactgtagaggcgtgtctccctctcgaactgacaaagaacgagtcttgtctcaatagtagggaaacttcattcattacaaacgggtcatgtcttgcttcaaggaagaccagcttcatgatggcactctgcttgtcttcaatctatgaggatcttaaaatgtaccaagtagagtttaagactatgaatgcgaagctcctgatggatccgaagcggcagatttttttggaccagaatatgttggcggtcattgacgaacttatgcaagctctcaatttcaattcagagacggttcctcagaaaagctccttggaagagccggacttctacaaaactaagatcaaattgtgtatcttgctccatgcattccggatacgcgccgtgaccattgatcgagtaatgtcctatttgaatgcaagctaa

SEQ ID NO: 33 - protein, 532 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - DNA, 984 bp, synthetic construct
  source 1..984: mol_type = other DNA
atgtgccaccagcaacttgtaataagttggttctcactggtcttccttgcgagcccgttggtagcaatctgggaactgaaaaaggacgtatatgtggtggagctcgattggtatcccgacgcacccggggagatggttgtcctcacctgtgataccccagaggaagacggtatcacttggaccttggatcagtcctccgaggtgcttggaagcggaaaaactctgacgatacaggtgaaagaattcggcgacgcgggacagtatacatgccacaaaggcggggaggtactgtcacattcactcctgctcttgcataaaaaggaggacgggatctggagcactgatattttgaaagaccaaaaagaaccaaaaaacaagaccttcctcaggtgtgaggccaagaattatagtggacgcttcacctgctggtggctgaccacaattagtactgacttgactttctcagtaaagagttccagggggagtagtgacccccaaggggtaacctgcggggccgcgactttgtcagcggaacgggtgcgaggggataataaggaatatgagtattcagtggagtgccaggaagactctgcatgtcccgcagcagaggaaagcctgccgatagaggtaatggttgacgccgtccataagctcaaatacgagaactacacaagctctttcttcatacgggacattattaagccagaccccccgaagaatcttcaacttaaaccgttgaaaaatagtcgacaagtggaagtcagttgggaatatccagacacctggagtacgccacacagctatttttccttgacattttgcgtccaggttcaggggaaatcaaagcgcgagaagaaggatcgagtctttacagacaagacgagtgcaacggtaatctgtaggaagaacgcaagcatttccgtcagagctcaggaccggtactatagcagttcatggtctgaatgggctagtgtaccttgcagt

SEQ ID NO: 35 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 36 - protein, 7 aa, synthetic construct
GGGGGGS

SEQ ID NO: 37 - DNA, 594 bp, synthetic construct
  source 1..594: mol_type = other DNA
cgaaatctgccagtcgctaccccggacccaggaatgtttccatgcctgcaccacagtcagaacctgctccgggcggtttccaacatgcttcagaaagcgcgccagacccttgaattttacccctgcacaagtgaagagatagaccatgaagatattaccaaggataaaacatcaactgtagaggcgtgtctccctctcgaactgacaaagaacgagtcttgtctcaatagtagggaaacttcattcattacaaacgggtcatgtcttgcttcaaggaagaccagcttcatgatggcactctgcttgtcttcaatctatgaggatcttaaaatgtaccaagtagagtttaagactatgaatgcgaagctcctgatggatccgaagcggcagatttttttggaccagaatatgttggcggtcattgacgaacttatgcaagctctcaatttcaattcagagacggttcctcagaaaagctccttggaagagccggacttctacaaaactaagatcaaattgtgtatcttgctccatgcattccggatacgcgccgtgaccattgatcgagtaatgtcctatttgaatgcaagctaa

SEQ ID NO: 38 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 39 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 41 - protein, 288 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 42 - protein, 13 aa, synthetic construct
IFDITNNLSIVIL

SEQ ID NO: 43 - protein, 105 aa, synthetic construct
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVK

SEQ ID NO: 44 - DNA, 20 bp, synthetic construct
  source 1..20: mol_type = other DNA
actggaagcacgaatgacag

SEQ ID NO: 45 - DNA, 20 bp, synthetic construct
  source 1..20: mol_type = other DNA
atgggccttcacacacatag

SEQ ID NO: 46 - DNA, 20 bp, synthetic construct
  source 1..20: mol_type = other DNA
tacccgaccaccatgtcctt

SEQ ID NO: 47 - DNA, 20 bp, synthetic construct
  source 1..20: mol_type = other DNA
atatagatctgcaactatgt